(19) United States
(12) Patent Application Publication
(10) Pub. No.: US 2012/0148628 A1
(43) Pub. Date: Jun. 14, 2012

(54) Compositions and methods for treatment of cancer

(76) Inventor: David S. Terman, Pebble Beach, CA (US)

(21) Appl. No.: 13/317,590

(22) Filed: Oct. 20, 2011

Related U.S. Application Data
(63) Continuation-in-part of application No. 12/860,699, filed on Aug. 20, 2010, now abandoned.
Application No. 12/860,699 is a division of application No. 10/428,817, filed on May 5, 2003, now abandoned.
(60) Provisional application No. 61/455,592, filed on Oct. 20, 2010.

(51) Int. Cl. A61K 38/16; A61P 35/00; A61P 37/04; C07K 19/00

(52) U.S. Cl. 424/282.1; 530/387.3; 530/350

(57) ABSTRACT

The present invention contemplates therapeutic constructs comprising superantigen homologues devoid of neutralizing antibodies in human sera conjugated recombinantly to costimulatory and tumor targeting molecules for treatment of cancer

### CROSS REFERENCE TO RELATED DOCUMENTS

The present application is a non-provisional of U.S. provisional patent application 61/455,592 filed on Oct. 20, 2010 and is a continuation in part of U.S. patent application Ser. No. 12/586,532 filed Sep. 22, 2009 which is a continuation in part of Ser. No. 12/276,941 filed Nov. 24, 2008 and Ser. No. 12/145,949 filed Jun. 25, 2008 both of which are divisionals of U.S. patent application Ser. No. 10/937,758 filed Sep. 8, 2004 which is a continuation of U.S. patent application Ser. No. 09/680,884 filed Aug. 30, 2000 which is a claims benefit of U.S. provisional patent application 60/151,470 filed Aug. 30, 1999.

The instant application is also a continuation in part of U.S. patent application Ser. No. 12/860,699 filed Aug. 20, 2010 which is a continuation of U.S. patent application Ser. No. 12/145,949 filed Jun. 25, 2008 issued as 7,803,637 on Jun. 4, 2010 which is a divisional of U.S. patent application Ser. No. 10/428,817 filed on May 5, 2003 (abandoned) and U.S. application Ser. No. 10/937,758 filed on Sep. 8, 2004 (abandoned) which is a continuation of U.S. application Ser. No. 09/650,884 filed on Aug. 30, 2000 (abandoned) which claims priority to provisional patent application 60/151,470 filed on Aug. 30, 1999.

Application Ser. No. 12/586,532 claims benefit to provisional application Ser. No. 61/215,906 filed May 11, 2009 and United State provisional application Ser. No. 61/211,227 filed Mar. 28, 2009 and U.S. provisional application Ser. No. 61/206,338 filed on Jan. 28, 2009 and U.S. provisional application Ser. No. 61/192,949 filed on Sep. 22, 2008 and PCT/US07/69869 filed May 29, 2007 and U.S. provisional application Ser. No. 60/809,553 filed on May 30, 2006 and U.S. provisional application Ser. No. 60/819,551 filed on Jul. 8, 2006 and U.S. provisional application Ser. No. 60/842,213 filed on Sep. 5, 2006 and U.S. patent application Ser. No. 10/428,817, filed May 5, 2003 and U.S. provisional application Ser. No. 60/438,686, filed Jan. 9, 2003 and U.S. provisional application Ser. No. 60/415,310, filed on Oct. 1, 2002 and U.S. provisional application Ser. No. 60/406,750, filed on Aug. 29, 2002 and U.S. provisional application Ser. No. 60/415,400, filed on Oct. 2, 2002 and U.S. provisional application Ser. No. 60/406,697, filed on Aug. 28, 2002 and U.S. provisional application Ser. No. 60/389,366, filed on Jun. 15, 2002 and U.S. provisional application Ser. No. 60/378,988, filed on May 8, 2002 and U.S. patent application Ser. No. 09/870,759 filed on May 30, 2001 and U.S. patent application Ser. No. 09/640,884 filed Aug. 30, 2000 and U.S. provisional patent application Ser. No. 60/151,470 filed on Aug. 30, 1999. All of these patent applications are incorporated in entirety with their references by reference.

### BACKGROUND OF THE INVENTION

1. Field of the Invention

The invention is in the fields of genetics and medicine and covers compositions and methods for treatment of cancer using heme in its native state or conjugated to tumor targeting agents or anti-tumor molecules such as superantigens and toxins. Heme may be used alone or together with free superantigens and heme oxygenase inhibitors to promote a tumoricidal effect.

2. Discussion of the State of the Art

The classic SE's (SEA-E) are known to induce antitumor activity versus solid tumors in animal models and humans (Terman D S et al., Clin Chest Med. 27:321-34 (2006)). Over the past decades, their clinical translation to human cancer has been hampered by the appearance of severe cardio-pulmonary toxicity associated with a high incidence of neutralizing antibodies in human sera (Holtfreter S et al., Infect Immun (2004) 72:4061-71; Alpaugh R K, et al., Clin Cancer Res 4:1903-14 (1998)). The latter are known to attenuate/abolish their T cell dependent anti-tumor effects.

In search of a superantigen with an improved therapeutic index without the encumbrance of neutralizing antibodies, we turned to the egc-SE's. The egc-SE's are a new generation of genetically linked staphylococcal enterotoxins (SE's) encoded by an operon of Staphylococcus aureus consisting of enterotoxins G, I, M, N, and O. They are associated with a strikingly low incidence of naturally occurring antibodies in human sera compared to the classic SE's. (Holtfreter S et al., supra; Jarraud S et al., J Immunol 166: 669-677 (2001)).

The egc SE's were used to treat 14 consecutive patients with stage 111B NSCLC and MPE and poor performance (median KPS 40). More than 80% of patients in this stage of advanced lung cancer present in poor condition (KPS<60) and are unsuitable for surgery or chemotherapy. They are thereby relegated to standard palliative measures such as talc insufflation or long term indwelling pleural catheter drainage for which the median survival is only two months Putnam J B Cancer (1999) 86:1992-1999; Burrows C M et al., Chest (2000) 117:73-78) We examined toxicity and efficacy of these agents in comparison with a similar group of 13 patients (median KPS 30) treated with talc insufflation. We also compared egc-SE's treatment to the marginal anti-tumor effects and clinical toxicity of classic SEA (Alpaugh R K et al., supra).

Intrapleurally delivered egc-SE's not only resolved MPEs in all 14 patients (12 cr, 2 pr) but also prolonged survival nearly 3 fold compared to talc insufflation (p=0.0023). Nine of 14 egc-SE-treated patients survived more than 6 months versus none of the 13 talc-treated patients. Two months posttreatment, KPS for the egc SE-treated group improved to a median of 70 from a pre-treatment median KPS of 40. In contrast, the pre-treatment median KPS of 30 in the talc-treated group declined to a posttreatment median KPS of 10 associated with a 93% mortality (p=<0.0001). In further contrast to talc-treated patients, the egc-SE treatment was carried out entirely in an outpatient setting without thoracotomy or indwelling catheter. Toxicity in the egc-SE-treated group was minimal and notably devoid of stage 3-4 toxic events frequently encountered with classic SEA treatment and respiratory distress seen in up to 10% of talc-treated patients. egc-SE's therefore simultaneously resolved MPEs and significantly improved survival whereas talc treatment merely resolved MPEs but conferred no survival benefit. Moreover, the negligible toxicity and clinical efficacy of the egc-SE's in this trial differed dramatically from the severe toxicity and minimal antitumor efficacy associated with the use of classic SEA in cancer patients (Alpaugh et al., supra). The egc-SE's thus appear to possess therapeutic activity exceeding that of talc insufflation and a therapeutic index/safety profile that is substantially broader than classic SEA.

egc-SE's are superantigens and use mechanisms similar those of the classic SE's to eradicate tumors. They induce robust vβ specific T cell proliferation in small doses (ED50=6.0 pg) and generate cytokines/nitrous oxide from PBMCs capable of killing a broad spectrum of tumors (Serrier, Terman, Lina et al., in press). The improved anti-tumor efficacy of the egc-SE's over the classic SEA in human cancer patients may be attributed to the strikingly lower incidence of neutralizing antibodies against egc-SE's in human sera. Such neutralizing antibodies against egc-SE's are present in only 5-10% of human sera compared to 80% for SEA wherein they attenuate/abolish SEA-mediated T cell proliferation ((Holtfreter S et al., supra; Alpaugh R K, et al., supra). The reduced toxicity of the egc-SE's may also be ascribed to the lower incidence of neutralizing antibodies, however an equally sound basis is the significantly lower levels of TH-1 cytokines generated by egc-SEG compared to classic SEA (Serrier, Terman, Lina et al., in press).

In the course of our in vitro and in vivo studies we noted surprisingly that as a single agent SEG possesses potent tumoricidal function with less toxicity than that of the egc family as a whole. Importantly, SEG also showed a low incidence of neutralizing antibodies in human sera compared to the classical SE's associated with the highest affinity for MHCII receptors of any known SEs. Thus SEG outcompetes the other egc SE's for MHCII receptors and thereby controls and modulates the tumoricidal activity and toxicity of the egc SE group as whole. Based on these features, SEG and its homologues are used as a backbone to be packaged together as fusion proteins with T cell costimulatory molecules and/or tumor targeting structures to provide a potent new treatment of cancer.

### SUMMARY OF THE INVENTION

The present invention contemplates therapeutic constructs comprising staphylococcal enterotoxin G homologues devoid of neutralizing antibodies in human sera conjugated recombinantly to costimulatory and tumor targeting molecules for treatment of cancer

### FIGURE LEGEND

FIG. 1: The vector with insertion sites for nucleic acids encoding SEG used to produce staphylococcal enterotoxin G reecombinantly is shown. This same vector among others is a model for other that can be used to introduce nucleic acids encoding SEG fused to costimulatory molecules and tumor targeting molecules.

### DESCRIPTION OF THE PREFERRED EMBODIMENTS

Staphylococcal enterotoxin G is encoded by the egc (enterotoxin gene complex) operon of staphylococcus aureus consists of 777 nucleotides encoding a mature protein of 233 (SEG) residues, 27,043 Da. It exhibits the ability to elicit T-cell proliferation with concomitant production of IL-2 and IFN-β. SEG shares 41-46% amino acid identity with other members of the SEB subfamily (SSA 46%, SEB 45%, SPEA 43%, SEC2-3 42%, SEC1-41%). While all SEB family members are known to interact with mVb8.2 TCR, SEG, shows substitutions in three key residues located in the conserved binding surface for mVβ8.2, resulting in an affinity for mVβ8.2 (KD ¼ 0.125 μM) 40 to 100-fold higher than that reported for other members of SEB subfamily, and the highest reported for any wild type SAg-TCR couple. SEG retains a fast dissociation rate characteristic of SAgs that allows sequential binding to several other TCR vβ molecules. Notably, SEG also has an extremely high affinity for MHCII-DR (KD ¼ 32 μM).

Inoculation of SEG into the right hind footpad of AKR/J mice showed the proliferation of mVb8.2 T-cells in regional lymph nodes was twice as great as in mice inoculated with SEC3; and the stimulation effect extended to lymph nodes from the left hind leg inoculated with PBS.

Unlike the classic SAgs, naturally occurring antibodies against SEG are rarely found in humans (Holtfreter S et al., supra (2004)). Moreover, the use of SEG in a clinical trial of 14 patients with advanced lung cancer was unattended by the toxicity seen previously in humans treated with other superantigens (Ren S, Terman D S et al., Chest (2004) 126 :1529-39; Alpaugh R K et al., supra (1998)). The latter effect may be attributed to the absence of neutralizing antibodies in human sera and SEG's more favorable cytokine profile.

Studies of recombinant egc-SE's demonstrated superantigenic activity, both in vitro and in vivo have revealed the following properties:
i. qRT-PCR (using both primers annealing to selected vβ nucleotide sequences and SYBR Green I reporter) showed vβ-dependent expansion of T cells by egc-SE's: (Seo K S, et al., J Transl Med 8: 1-9 (2010))
   i. SEG: vβ3,12,13A,13B,14,15;
   ii. SEI, vβ1,5,6,23;
   iii. SEM, vβ6,8,9,8,21;
   iv. SEN, vβ7,8,9,17;
   v. SEQ, vβ5
ii. Nitrous oxide and TNFa dependent killing of a broad range of tumor cells including human lung, head and neck, colon and breast carcinoma cell lines comparable to SEA (Serrier, Terman, Lina et al., supra)
iii. Potent T cell mitogenicity comparable to SEE using doses in the superantigenic range (Jarraud et al., supra)
iv. TH-1 cytokine release from human PBMCs in quantities comparable to SEA. (Serrier, Terman, Lina et al., supra)
v. wtSEG, SEG-R47 (wtSEG with _{lys}47_{arg} substitution) and control SEA induced T cell proliferation with ED₅₀s as follows: SEA=<1 fM; SEG=15 fM; and SEG-R47=4 nM each well within superantigenic range for wtSEs and SE homologues.
vi. NZW rabbits given an intravenous dose of SEG (20 pg/kg) showed mild pyrexia and tachycardia of short duration.
vii. egc-SEs used in clinical trial in patient with advanced lung cancer simultaneously induced a significant survival benefit and resolution of all pleural effusion compared to talk insufflation which induced only remission of pleural effusion but no survival benefit.

In the course of analyzing these data, noted the surprising and highly relevant features of SEG that recommend it as a single agent in place of the egc SE family as a whole was noted. These notable properties of SEG include:
i. the broadest vβ TCR stimulation profile of all egc-SE's and powerful T cell mitogenic activity (Seo K S, et al., supra)
ii. a broad range of nitrous oxide/TNFα dependent tumor cell cytotoxicity comparable to SEA (Serrier, Terman, Lina et al., supra).
iii. the lowest levels of any egc SE of toxicity-inducing TH-1 cytokines compared to classic SEA. TNFα (p=0.004) (Serrier, Terman, Lina et al., supra)
   a. IFN-γ (p<0.001),
   b. IL-2 (p<0.001),
   c. IL-17 (p<0.001)
   d. GM-CSF (p=0.07).
iv. iv. a low incidence of efficacy-disrupting neutralizing antibodies in human sera, 5-10% for SEG compared to 60-80% for SEA (Holtfreter S et al., supra.)
v. v. the highest binding affinity to MHCII receptors (KD 0.125 uM by SPR) of any known SE (Fernandez, M M et al., Proteins 68:389-402 (2007).
vi. mild constitutional toxicity in vivo (see above)

Notably, SEG generates lower levels of toxicity-inducing cytokines while still retaining a degree and breadth of nitrous oxide dependent tumor cytotoxicity comparable to that of SEA. In this context, SEG shows the highest affinity for TCR of any SE leading to its potent and widespread stimulation of mouse Vβ8.2 T-cells in vivo when compared with other members of the SEB subfamily. Importantly, SEG possesses the highest binding affinity (Kd) for MHCII cells of any known superantigen (Fernandez, M M et al., supra) enabling it to outcompete other SEs for MHCII binding sites on APCs in vivo. As such SEG may interdict the cytokine generating ability of coadministered egcSEs as used clinically (Ren S, Terman D S et al., supra) and thereby control and modulate both toxicity and tumor killing profile of the egcSE family as a whole in vivo. This may explain the negligible toxicity observed in the clinical trial (Ren S, Terman D S et al., (2004)); Terman D S et al., Clin Chest Med. 27:321-34 (2006)) and the lower degree of cardiopulmonary toxicity noted in sepsis caused by multiple egc-SE's (including SEG) compared to that induced by SEA (Ferry T, et al., Clin Microbiol Infect 14: 546-554 (2008)).

SEG and its homologues described below are used as free polypeptide or fused to costimulatory molecules and or tumor specific targeting molecules as described below. These agents are tested for anti-tumor activity as described herein in the following sections: “Pharmaceutical Compositions and Administration”, “Injectable Formulations”, “Animal and human testing”, “Tumor Models and Procedures for Evaluating Anti-Tumor Effects Studies”, Examples 1 and 2.

Wild Type egc-SEs, egc SE Homologues, egc SE Fusion Proteins and Related SEs Devoid of Preexistent Neutralizing Antibodies with Mutations of SE MHC Class II Binding Sites.

Up to 80% of human sera contain preexisting neutralizing antibodies specific for SEA, SEB, SEC1-3 or SpeA that are capable of abolishing the T cell mitogenic function of these agents. This has been a major impediment to the successful use of SEs for cancer therapy. Several new naturally occurring staphylococcal enterotoxins and streptococcal pyrogenic exotoxins have been identified which do not exhibit preexistent, neutralizing tumor associated antibodies in human sera. Among these are the egc-SE family that includes SEG, SEI, SEM, SEN and SEQ. These genetically-linked SEs are weakly transcribed and spend most of their life cycle intracellularly embedded in staphylococcal operon. Recently SEG has been added to the evolutionarily related SE subgroup comprising SEB, SSA, SpEA, SEC1-3 and SER. Moreover SEI and SEM of the egc family have been found to be related to another evolutionarily related subgroup SEA, SEE, SED. The present inventions contemplates the use of these new wild type SEs and SEG and its homologue in particular, without preexistent neutralizing antibodies in human sera for use in therapy of carcinoma. In addition, the MHCII binding site(s) is(are) modified by amino acid substitutions which reduces the affinity of these molecules for the MHCII. This leads to attenuation of their cytokine-mediated toxicity such as tachycardia, hypotension and fever while preserving sufficient T cell mitogenic function to support carcinoma killing.

An additional embodiment envisions that SEG and related SEs are more effective when key site(s) for MHCII binding are deleted or substituted. Residues adjacent to the hydrophobic loop or polar binding pocket that contact HLA-DR or residues at sites that can indirectly alter the structure of the hydrophobic loop or polar pocket can reduce MHCII binding. Such mutation at any of several sites results in reduction of MHCII affinity and reduction in toxicity of SEG. The amino acid sequence and structural analysis of SEG support a SEB-like interaction with a chain of MHCII since the most important residues of SEB that contact DR1 molecule are conserved. Thus, residues Gln44, Phe45, Leu46, Tyr87, and Tyr110 (SEG numbering) that make hydrogen bond contacts with MHC-DR1 are conserved in SEG. In addition, most of the hydrophobic residues stabilizing the SEB-DR1 interaction, Phe45, Leu46, and Phe48, are present in SEG, or conservatively substituted. The salt bridge provided for Glu65 with the surrounding residues Y87 and Y110 that make H-bonds are strictly conserved and other key contact residues as Lys76 and Lys208 are also conserved.

A motif consisting of a leucine in a reverse turn is conserved among bacterial superantigens and provides the key determinant (hydrophobic or otherwise) for binding HLA-DR. Like SEB, the SEG toxin sequence is preferably altered at the hydrophobic loop or polar binding pocket. The hydrophobic region of the binding interface between SEG or SEB and the HLA-DR1 molecule consists of SEB residues 44-47, located in a large reverse turn connecting β-strands 1 and 2 of SEB and SEG. These residues appear to make strong electro-static interactions with DRα through their backbone atoms. The mutation of L45 to an arginine reduced overall HLA-DR1 binding greater than 100-fold, attributable to the less energetically favorable insertion of a highly charged residue into a hydrophobic depression on the DR1 molecule. SEG and SEB L45 is the most extensively buried residues in the DR1 interface. The leucine is conserved among the bacterial superantigens and provides the hydrophobic structural element requisite for surface complementarity with DR1. The mutation of L45 to an arginine reduced overall HLA-DR1 binding greater than 100-fold, attributable to the less energetically favorable insertion of a highly charged residue into a hydrophobic depression on the DR1 molecule. SEG and SEB L45 is the most extensively buried residues in the DR1 interface. The leucine is conserved among the bacterial superantigens and provides the hydrophobic structural element requisite for surface complementarity with DR1. These considerations indicate that substitution at this SEG L47 to an arginine will reduce MHCII binding up to 100 fold and as such is the preferred substitution site.

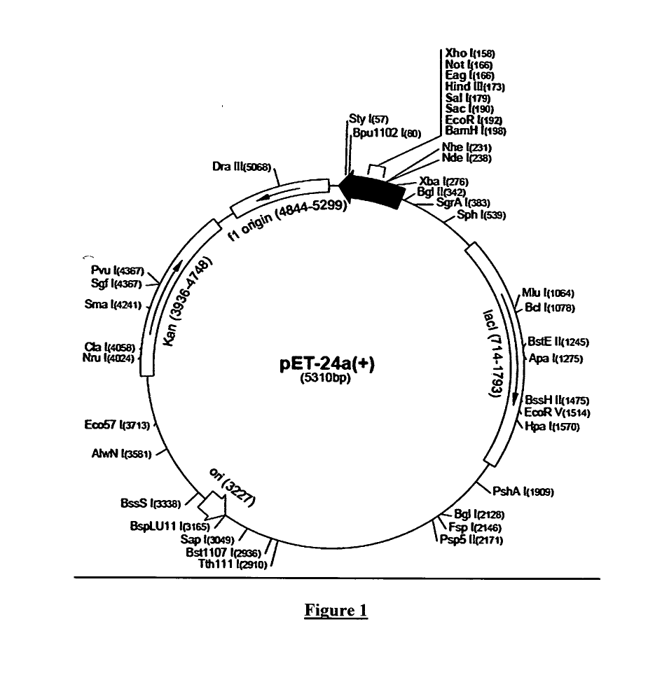

Indeed, just such a substitution was effected herein using site directed mutagenesis and the vector described in FIG. 1. The resulting molecule SEGₗₑᵤ47_{arg} showed T cell mitogenicity ED₅₀=4 nM or three logs less than wild type SEG indicating a reduction in MHCII binding. This modified SEGₗₑᵤ47_{arg} is the preferred form of SEG alone or fused recombinantly to a costimulatory molecule and/or a tumor specific targeting molecule as described below.

The number of SEG residues which can be altered can vary, preferably the number can be 1-2, more preferably 2-3, and most preferably 3-4, or more with the limitation being the ability to analyze by computational methods the consequences of introducing such mutations. The residues which can be altered can be within 5 amino acid residues of the central Leucine of the hydrophobic loop (such as L46 of SEB or L45 of SEG), or within 5 residues of one of the amino acid residues of the polar binding pocket that can contact HLA-DR, (such as E67, Y89, or Y115 of SEG or SEB), more preferably, within 3 amino acid residues of the central Leucine of the hydrophobic loop (such as L45 of SEB, L46 of SEG), or within 3 residues of one of the amino acid residues of the polar pocket that can contact HLA-DR, (such as E67, Y89, or Y115 of SEB), and most preferably, the central Leucine of the hydrophobic loop (such as L45 of SEB), or one of the amino acid residues of the polar binding pocket that can contact HLA-DR, (such as E67, Y89, or Y110 of SEB or SEG in the Table below). The residues can be changed or substituted to alanine for minimal disruption of protein structure, more preferably to a residue of opposite chemical characteristics, such as hydrophobic to hydrophilic, acidic to neutral amide, most preferably by introduction of a residue with a large hydrated side chain such as Arginine or Lysine. In addition, side chains of certain non-conserved receptor-binding surfaces, can also be altered when designing superantigen toxins with low binding affinities. These residues can include Y94 of SEB and structurally equivalent residues of SEG. In addition amino acids in SER, SSA, SEC1-3, SpeA identical or homologous amino acids and located at the identical or similar MHC-DR binding positions as SEB and SEG above can be modified in a similar fashion to achieve a reduction in MHCII binding. These amino acids and their locations are shown in the Table below (SEQ ID NOS: 1-8).

Additionally, mutation of residue Y89 in SEB and SEG results in greater than 100-fold reduction in DR1 binding. Substitution of SEB Y115 with alanine also resulted in greater than 100-fold reduction of binding. The K39 side chain of DRα forms a strong ion-pair interaction with the SEB E67 carboxylate group and hydrogen bonds with the hydroxyl groups of Y89 and Y115. Substitution of SEB E67 by glutamine reduced binding affinity by greater than 100-fold, reflecting the replacement of the strong ionic bond with a weaker hydrogen bond. Mutations in these regions of SEG would be expected to induce comparable reduction of MHCII affinity and are therefore useful in the present invention.

The regions of HLA DR1 that contact SEB and SEB consists exclusively of α subunit surfaces with the main binding regions consisting principally of two structurally conserved surfaces located in the N-terminal domains: a polar binding pocket derived from three β-strand elements of the β-barrel domain and a highly solvent-exposed hydrophobic reverse turn. The binding pocket of SEG and SEB contains residues E67 (E=Glutamic acid), Y89 (Y=Tyrosine) and Y115 (Y=tyrosine), and binds K39 (K=Lysine) of the DRα subunit while the hydrophobic region consists of a leucine and flanking residues that make several contacts with the HLA DRα chain.

SEG, SEGₗₑᵤ47_{arg}, SER, SEB, SEC1-3 and SpeA are prepared recombinantly by methods described herein and in PCTUS05/022638 incorporated by reference. The method of introduction of preferred L45 mutation and other mutations in MHCII binding sites in SEG described above can also be carried out in identical homologous sites for SEB, SER, SSA, SEC1-3 and SpeA using site directed mutagenesis as described in U.S. patent application Ser. No. 10/428,817 incorporated by reference. These mutations produce a significant reduction in the cytokine-inducing toxicity of SEG such as tachycardia, hypotension and fever while retaining sufficient mitogenic activity to support tumoricidal activity of the molecule versus carcinomas. SEG, SEGₗₑᵤ47_{arg} and related mutants with deletion of MHCII binding regions of SEG or other wild type superantigens, superantigen variants and fusion proteins similarly modified are tested in the section on “Tumor Models and Procedures for Evaluating Anti-Tumor Effects Studies” and in Human Patients in Examples 1 and 2.

With respect to SEI, crystallographic studies have shown that staphylococcal and streptococcal superantigens belonging to the zinc family bind to a high affinity site on the MHCII β-chain. These zinc-dependent superantigens achieve promiscuous binding to MHC by targeting conservatively substituted residues of the polymorphic β-chain. The interaction of the β1-β2 turn of DR1 is restricted to Thr21β, which makes a single van der Waals contact with Asn171 of SEI. A zinc ion is observed to bridge HLA-DR1 and SEI by tetrahedrally coordinating three ligands from the SEI (His169 from strand β12 and His207 and Asp209 from strand β15) with one ligand from the MHC β1 α-helix (His81β). Histidines 169 and 207 of SEI bind through their Nδ1 and N [symbol] 2 atoms, respectively, whereas His81β of HLA-DR1 binds through its Nδ1 atom.

All three SAg residues that coordinate zinc in the SEI-DR1 complex (His169, His207, and Asp209) are structurally conserved in the SPEC.MBP.DR2a complex, although SEI His 169 is from strand β12 of SEI, whereas the corresponding SPEC residue (His167) is from strand β13. SEI residues His207 and Asp209 are identical across the entire family of zinc-dependent SAGs, and His 169 is present in 13 of 16 members, if one includes SPEC. Notably, SEI Asn98, like zinc coordinating residues His207 and Asp209, is strictly conserved in all zinc-dependent SAgs. In addition to Thr77β and His81β, zinc-dependent SAgs form key interactions with several other conserved, or conservatively substituted, residues of the polymorphic DR β-chain, in particular Glu69β and Asp76β.

In the SEs listed in the Table below (SEQ ID NOS: 9-23), the present invention contemplates substitutions of any of the key conserved amino acids involved in the MHCII binding. A single substitution is sufficient. The key substitutions are at the His169, His207, and Asp209 of SEI and its equivalent in any of the other SEs shown below aligned with these SEs. These substitution are introduced using site directed mutagenesis and related methodology as described in U.S. patent application Ser. No. 10/428,817 incorporated by reference. These mutations produce a significant reduction in the cytokine-related toxicity of SEG such as tachycardia, hypotension and fever while retaining sufficient mitogenic activity to support tumoricidal activity of the molecule versus carcinomas. This and other wild type superantigens, superantigen variants and fusion proteins are tested in the section on the section on Tumor Models and Procedures for Evaluating Anti-Tumor Effects Studies pages and in Human Patients in Example 1 of this application.

These superantigen variants and mutants including SEG and SEGₗₑᵤ47_{arg} or other wild type superantigens, superantigen variants and fusion proteins similarly modified are fused to a polypeptide fusion partner such as costimulatory molecules and tumor specific antibodies, antibody fragments, tumor receptors or tumor ligands and additional polypeptide fusion partners using recombinant methods established in the art and provided in provided in U.S. Pat. No. 7/117,822 issued Jul. 17, 2010. SEG, SEGₗₑᵤ47_{arg}. They are tested in the section on “Tumor Models and Procedures for Evaluating Anti-Tumor Effects Studies” and in Human Patients in Examples 1 and 2.

### Fusion Partners for Native SEs or SE Homologues

Antibodies

In another embodiment, fusion protein partners for SAg or preferably SEG or SEG homologues include tumor specific antibodies, preferably F(ab′)₂, Fv or Fd fragments thereof, that are specific for antigens expressed on the tumor. In another embodiment, a fusion partner is a polypeptide ligand for a receptor expressed on tumor cells. These antibodies, fragments or receptor ligands may be in the form of synthetic polypeptides. The nucleic acid form of the antibody is envisioned which is useful as a fusion construct with the SAg DNA. Such a fusion protein is prepared using a fusion gene comprising nucleic acids encoding the SEG or SEG homologue and the tumor targeting structure. Methodology is described in the art and in Example 2. The vector for recombinant SEG production described herein is useful for this purpose. The same methodology can be used to fuse costimulatory molecules to SEG or an SEG homologue or any useful SAg or SAg homologue described herein.

One advantage of certain antibody constructs of the present fusion polypeptides is prolonged half-life and enhanced tissue penetration. Intact antibodies in which the Fc fragment of the Ig chain is present will exhibit slower blood clearance than their Fab′ fragment counterparts, but a fragment-based fusion polypeptide will generally exhibit better tissue penetrating capability.

Preferentially, the tumor targeting structure in the SEG, SEG homologue conjugate (e.g., tumor specific antibody, Fab or single chain Fv fragments or tumor receptor ligand) has a greater affinity for the tumor than the SAg in the conjugate has for the class II molecule thus preventing the SAg from binding all MHC class II receptors and favoring binding of the conjugate to the tumor. In the case of SEB, the dominant epitope for neutralizing antibodies 225-234 is recombinantly or biochemically bound to the tumor targeting molecule e.g., tumor specific antibodies, Fas or Fv fragments. In so doing, it sterically interferes with the recognition of the dominant epitope by preexisting antibodies.

To further enhance the affinity of the tumor specific antibody in the conjugate for tumor cells in vivo, tumor specific antibodies are used which are specific for more than one antigenic structure on the tumor, tumor stroma or tumor vasculature or any combination thereof. The tumor specific antibody or F(ab′)₂, Fab or single chain Fv fragments are mono or divalent like IgG, polyvalent for maximal affinity like IgM or chimeric with multiple tumor (tumor stroma or tumor vasculature) specificities. Thus, when the SE or SPE-MoAb conjugate is administered in vivo, it will preferentially bind to tumor cells rather than to endogenous SE antibodies or MHC class II receptors.

To reduce affinity of the SE-mAb conjugate for endogenous MHC class II binding sites, the high affinity Zn⁺⁺ dependent MHC class II binding sites in SEA, SEC2, SEC3, SED, SPEA, SPEC, SPEG, SPEH, SMEZ, SMEZ2, M. arthritides are deleted or replaced by inert sequence(s) or amino acid(s). These structural alterations in SE or SPEA reduce the affinity for MHC class II receptors from a K_{d} of 10⁻⁷ or 10⁻⁸ to 10⁻⁵. SEB, SEC and SSA and other SEs or SPEs do not have a high affinity Zn++ dependent MHC class II binding site but have multiple low affinity MHC class II binding sites (K_{d} 10⁻⁵-10⁻⁷). In these cases, alteration of the MHC class II binding sites is not always necessary to further reduce affinity for MHC class II receptors; at the very least mutation of one or two of the low affinity MHC class II binding sites will suffice in most instances.

Most importantly, tumor specific antibodies, Fab, F(ab′)₂ or single chain Fab or Fv fragments in the SAg-mAb conjugate have a higher affinity for tumor antigens (K_{d} 10⁻¹¹-10⁻¹⁴ or lower) than for the SAg has for MI-IC class II binding sites (K_{d} 10⁻⁵ to 10⁻¹¹) and its dominant epitope has for SAg specific antibodies (K_{d} 10⁻⁷ to 10⁻¹¹). In this way, the conjugate will bind preferentially to the tumor target in vivo rather than preexisting antibodies or MHC class II receptors.

Antibody fragments are obtained using conventional proteolytic methods. Thus, a preferred procedure for preparation of F(ab′)₂ fragments from IgG of rabbit and human origin is limited proteolysis by the enzyme pepsin. Rates of digestion of an IgG molecule may vary according to isotype; conditions are chosen to avoid significant amounts of completely degraded IgG as is known in the art.

Fab fragments include the constant domain of the light chain (CO and the first constant domain (C_{H1}) of the heavy chain. Fab′ fragments differ from Fab fragments by the addition of a few residues at the C-terminus of C_{H1} domain including one or more cysteine(s) from the antibody hinge region. F(ab′)₂ fragments were originally produced as pairs of Fab′ fragments that have hinge cysteines between them. Other chemical couplings of antibody fragments are also known.

An “Fv” fragment is the minimum antibody fragment that contains a complete antigen-recognition and binding site. This region consists of a dimer of one heavy chain and one light chain variable domain in tight, con-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the scFv to form the desired structure for antigen binding.

The following documents, incorporated by reference, describe the preparation and use of functional, antigen-binding regions of antibodies: U.S. Pat. Nos. 5,855,866; 5,965,132; 6,051,230; 6,004,555; and 5,877,289.

“Diabodies” are small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described in EP 404,097 and WO 93/11161, incorporated herein by reference. “Linear antibodies”, which can be bispecific or monospecific, comprise a pair of tandem Fd segments (V_{H}-C_{H1}-V_{H}-C_{H1}) that form a pair of antigen binding regions.

An antibody fragment may be further modified to increase its half-life by any of a number of known techniques. Conjugation to non-protein polymers, such PEG and the like, is also contemplated

The antibody fusion partner for use in the present invention may be specific for tumor cells, tumor stroma or tumor vasculature. Antigens expressed on tumor cells that are suitable targets for mAb-SAg fusion protein therapy include erb/neu, MUC1, 5T4 and many others. Antibodies specific for tumor vasculature bind to a molecule expressed or localized or accessible at the cell surface of blood vessels, preferably the intratumoral blood vessels, of a vascularized tumor. Such molecules include endoglin (TEC-4 and TEC-11 antibodies), a TGFβ. receptor, E-selectin, P-selectin, VCAM-1, ICAM-1, PSMA, a VEGFNPF receptor, an FGF receptor, a TIE, an αᵥβ₃ integrin, pleiotropin, endosialin and MHC class II proteins. Such antibodies may also bind to cytokine-inducible or coagulant-inducible products of intratumoral blood vessels. Certain preferred agents will bind to aminophospholipids, such as phosphatidylserine or phosphatidylethanolamine.

A tumor cell-targeting antibody, or an antigen-binding fragment thereof, may bind to an intracellular component that is released from a necrotic or dying tumor cell. Preferably such antibodies are mAbs or fragments thereof that bind to insoluble intracellular antigen(s) present in cells that may be induced to be permeable, or in cell ghosts of substantially all neoplastic and normal cells, but are not present or accessible on the exterior of normal living cells of a mammal.

Anti-tumor stroma antibodies bind to a connective tissue component, a basement membrane component or an activated platelet component; as exemplified by binding to fibrin, RIBS (receptor-induced binding site) or LIBS (ligand-induced binding site).

Fusion proteins include linkers or spacers that are incorporated into the recombinant fusion construct. Numerous types of disulfide-bond containing linkers are known that can be successfully employed to fuse the SAg to an antibody or fragment, certain linkers are preferred based on differing pharmacological characteristics and capabilities. For example, linkers that contain a disulfide bond that is sterically “hindered” are preferred, due to their greater stability in vivo, thus preventing release of the SAg moiety prior to binding at the site of action.

The SEG or SEG homologue-tumor targeting fusion proteins described above are administered parenterally, intratumorally, intrathecally (e.g., intraperitoneally, intrapleurally) by infusion or injection in conventional or sustained release vehicles in dosages of 0.01 ng/kg to 100 μg/kg every 3-7 days as comprehensively described herein in sections on Tumor Models and Examples 1 and 2.

### Coaguligand

SAgs may be conjugated to, or operatively associated with, polypeptides that are capable of directly or indirectly stimulating coagulation, thus forming a “coaguligand” (Barinaga M et al., Science 275:482-4 (1997); Huang X et al., Science 275:547-50 (1997); Ran S et al., Cancer Res 1998 Oct. 15; 58(20):4646-53; Gottstein C et al., Biotechniques 30:190-4 (2001)).

In coaguligands, the antibody may be directly linked to a direct or indirect coagulation factor, or may be linked to a second binding region that binds and then releases a direct or indirect coagulation factor. The ‘second binding region’ approach generally uses a coagulant-binding antibody as a second binding region, thus resulting in a bispecific antibody construct. The preparation and use of bispecific antibodies in general is well known in the art, and is further disclosed herein.

Coaguligands are prepared by recombinant expression. The nucleic acid sequences encoding the SAg are linked, in-frame, to nucleic acid sequences encoding the chosen coagulant, to create an expression unit or vector. Recombinant expression results in translation of the new nucleic acid, to yield the desired protein product.

Where coagulation factors are used in connection with the present invention, any covalent linkage to the SAg should be made at a site distinct from the functional coagulating site. The compositions are thus “linked” in any operative manner that allows each region to perform its intended function without significant impairment. Thus, the SAg binds to and stimulates T cells, and the coagulation factor promotes blood clotting.

Preferred coagulation factors are Tissue Factor (“TF”) compositions, such as truncated TF (“tTF”), dimeric, multimeric and mutant TF molecules. tTF is a truncated TF that is deficient in membrane binding due to removal of sufficient amino acids to result in this loss. “Sufficient” in this context refers to a number of transmembrane amino acids originally sufficient to insert the TF molecule into a cell membrane, or otherwise mediate functional membrane binding of the TF protein. The removal of a “sufficient amount of transmembrane spanning sequence” therefore creates a tTF protein or polypeptide deficient in phospholipid membrane binding capacity, such that the protein is substantially soluble and does not significantly bind to phospholipid membranes. tTF thus substantially fails to convert Factor VII to Factor VIIa in a standard TF assay yet retains so-called catalytic activity including the ability to activate Factor X in the presence of Factor VIIa.

U.S. Pat. No. 5,504,067, specifically incorporated herein by reference, describes tTF proteins. Preferably, the TFs for use herein will generally lack the transmembrane and cytosolic regions (amino acids 220-263) of the protein. However, the tTF molecules are not limited to those having exactly 219 amino acids.

Any of the truncated, mutated or other TF constructs may be prepared in dimeric form employing the standard techniques of molecular biology and recombinant expression, in which two coding regions are arranged in-frame and are expressed from an expression vector. Various chemical conjugation technologies may be employed to prepare TF dimers. Individual TF monomers may be derivatized prior to conjugation.

The tTF constructs may be multimeric or polymeric, which means that they include 3 or more TF monomeric units. A “multimeric or polymeric TF construct” is a construct that comprises a first monomeric TF molecule (or derivative) linked to at least a second and a third monomeric TF molecule (or derivative). The multimers preferably comprise between about 3 and about 20 such monomer units. The constructs may be readily made using either recombinant techniques or conventional synthetic chemistry.

TF mutants deficient in the ability to activate Factor VII are also useful. Such “Factor VII activation mutants” are generally defined herein as TF mutants that bind functional Factor VII/VIIa, proteolytically activate Factor X, but substantially lack the ability to proteolytically activate Factor VII.

The ability of such Factor VII activation mutants to function in promoting tumor-specific coagulation is requires their delivery to the tumor vasculature and the presence of Factor VIIa at low levels in plasma. Upon administration of a conjugate of a Factor VII activation mutant, the mutant will localize within the vasculature of a vascularized tumor. Prior to localization, the TF mutant would be generally unable to promote coagulation in any other body sites, on the basis of its inability to convert Factor VII to Factor VIIa. However, upon localization and accumulation within the tumor region, the mutant will then encounter sufficient Factor Vila from the plasma in order to initiate the extrinsic coagulation pathway, leading to tumor-specific thrombosis. Exogenous Factor VIIa could also be administered to the patient to interact with the TF mutant and tumor vasculature.

Any one or more of a variety of Factor VII activation mutants may be prepared and used in connection with the present invention. The Factor VII activation region generally lies between about amino acid 157 and about amino acid 167 of the TF molecule. Residues outside this region may also prove to be relevant to the Factor VII activating activity. Mutations are inserted into any one or more of the residues generally located between about amino acid 106 and about amino acid 209 of the TF sequence (WO 94/07515; WO 94/28017; each incorporated herein by reference).

A variety of other coagulation factors may be used in connection with the present invention, as exemplified by: the agents set forth below. Thrombin, Factor V/Va and derivatives, Factor VIII/VIIIa and derivatives, Factor IX/IXa and derivatives, Factor X/Xa and derivatives, Factor XI/XIa and derivatives, Factor XII/XIIa and derivatives, Factor XIII/XIIIa and derivatives, Factor X activator and Factor V activator may be used in the present invention.

These conjugates are administered parenterally by infusion or injection in dosages of 0.01 ng/kg to 100 μg/kg.

### Cytokines as Fusion Partners

Cytokines are an effective partner for SAgs. Various cytokines, such as IL-2, IL-3, IL-7, IL-12, and IL-18, may be used.

A preferred fusion polypeptide comprises a SAg fused to anti-apoptotic cytokines.

SAg stimulation of T cells can result in activation-driven cell death. Several cytokines and bacterial lipopolysaccharide (LPS) are known to interfere with this process (Vella et al., Proc. Natl. Acad. Sci. 95: 3810-3815 (1998)). IL-3, IL-7, IL-15 and IL-17 prevent SAg-stimulated T cells from undergoing apoptosis in vivo and in vitro. In addition, because of their ability to promote selective proliferation by Th₁ T cells, IL-12 and IL-18 are desirable. IL-18 is preferred for intratumoral injection because it induces tumor suppressive cytokines IFNγ and TNFα and IL-1β, and rescues cytotoxic T cells from apoptosis.

Accordingly, SAg-mAb conjugate as described above is fused recombinantly to the extracellular domains of at least one cytokine from a group consisting of IL-2. IL-7 or IL-3 or IL-12 or IL-15 or IL-17 or IL-18. Other anti-T cell apoptosis agents such as LPS preparations of low virulence or a lipid A component (modified to induce less toxicity) are also effective antiapoptotic agents when conjugated biochemically to the SAg-MoAb (or F(ab′)₂, Fab, Fd or single chain Fv fragments) conjugate or if administered concomitantly with the SAg. Nucleic acids encoding the cytokine of choice is fused in frame with nucleic acids encoding the SAg. These conjugates are administered parenterally, intrathecally and/or intratumorally by infusion or injection in dosages of 0.01 ng/kg to 100 μg/kg.

### Costimulatory Molecules as Fusion Partners

Superantigens Conjugated to OX40L or 4-1BBL

An additional embodiment comprises a fusion polypeptide consisting of SEG or an SEG homologue fused recombinantly to a potent costimulatory molecule, preferably the ECD of a transmembrane costimulatory protein. Examples of such costimulatory molecules are the OX-40 ligand (Godfrey et al., J. Exp. Med. 180: 757-762 (1994); Gramaglia I et al., J. Immunol. 161: 6510-6517 (1998); Maxwell J R et al., J. Immunol. 164: 107-112 (2000) or 4-1BB ligand (Kown B S et al., Proc. Natl. Acad. Sci. USA 86:1963-67 (1989); Shuford W W et al., J. Exp. Med. 186: 47-55 (1997) and CD-38 (Jackson D G et al., J. Immunol. 144: 2811-2817 (1990); Zilber et al., Proc. Nat'l Acad. Sci. USA 97: 2840-2845 (2000). The preparation of such fusion proteins is achieved by recombinant methods in which nucleic acids encoding SAgs are fused in frame to nucleic acids encoding the ECD of the costimulatory molecule such as OX-40L (Godfrey et al., J. Exp. Med. 180:757-762 (1994)) or 4-1BBL (Goodwin et al. Eur. J. Immunol. 23: 2631-2641 (1993); Melero I. et al., Eur. J. Immunol. 28: 1116-1121 (1998)).

SEG or SEG homologue with a deletion in a key MHCII binding site are preferred SAg partners for the cositmulatory molecules. However, if another SAg or SAg homologue is used as a partner, it is preferred to delete from the conjugates or fusion polypeptides of the present invention any SAg epitope that binds to SAg-specific antibodies such as preexisting or natural or neutralizing antibodies. Such epitopes are deleted or substituted by Ala or by amino acid sequences not recognized by preexisting host antibodies. For example, a dominant epitope of SEB that is recognized by anti-SEB antibodies is the sequence at residues 225-234 (Nishi et al., J. Immunol. 158: 247-254 (1997). An epitope of SEA that is recognized by anti-SEA antibodies is the sequence at residues 121-149 (Hobieka et al., Biochem. Biophys. Res. Comm. 223: 565-571 (1996). Alternatively, to avoid issues with such preexisting immunity, SAgs such as the egc-SEs or the newly recognized SAg and their homologues such as SEP, SEQ, SER, SEU, YPM or C. perfringens toxin A to which humans do not have preexisting antibodies such as the egc-SE's are selected. YPM, in addition, a natural RGD domain which gives it tumor localizing properties. These SE may also be modified to reduce their toxicity by altering their MHC class II binding affinity as described herein for SEG. SEA's high affinity MHCII binding and associated toxicity was reduced substantially by implementing a D227A-high affinity Zn++ dependent binding site substitution.

Preferably, the tumor targeting structure in SAg conjugate (e.g., tumor specific antibody or fragment, or a tumor receptor ligand) has greater affinity for the tumor than the affinity of the SAg in the conjugate for the MHC class II molecule thus preventing the SAg from binding “promiscuously” to all MHC class II molecules receptors and favoring binding to the tumor.

To further enhance the affinity of the tumor specific antibody in the SAg-costimulatory molecule fusion polypeptide for tumor cells in vivo, one may select a tumor specific antibody that is specific for more than one antigenic structure in the tumor, the tumor stroma or the tumor vasculature (or any combination). The tumor specific antibody or antigen-binding fragment thereof can be made mono or divalent (like IgG), polyvalent like IgM to increase avidity or chimeric with multiple tumor specificities as described above. Thus, when the SAg-mAb conjugate is administered in vivo, it will preferentially bind to tumor cells rather than to endogenous anti-SAg antibodies or MHC class II receptors.

To reduce affinity of the SAg-mAb conjugate for endogenous MHC class II binding sites, the high affinity Zn⁺⁺ dependent MHC class II binding site present in a number of SAgs (SEA, SEC2, SEC3, SED, SPEA, SPEC, SPEG, SPEH, SMEZ, SMEZ2, M. arthritides SAg) is deleted or replaced by an “inert” sequence(s) or amino acid. Such structural alterations in SE or SPEA are known to reduce the affinity for MHC class II from a K_{d} of 10⁻⁷ or 10⁻⁸ to a K_{d} of 10⁻⁵. SEB, SEC and SSA and other SAgs do not have such a high affinity Zn⁺⁺-dependent MHC class II binding site but have multiple low affinity MHC class II binding sites (K_{d} of 10⁻⁵-10⁻⁷). In these cases, alteration of the MHC class II binding sites is not always necessary to further reduce affinity for MHC class II; mutation of one or two of the low affinity MHC class II binding sites will suffice in most instances.

Most importantly, tumor specific antibodies or their fragments in a SAg-mAb conjugate have higher affinities for tumor antigens (K_{d} of 10⁻¹¹-10⁻¹⁴ or lower) than (a) the affinity of the SAg for MHC class II binding sites (K_{d} 10⁻⁵ to 10⁻⁷) or (b) the affinity a dominant SAg epitope for a SAg-specific antibody(K_{d} 10⁻⁷ to 10⁻¹¹). Because of this, the conjugate will bind preferentially to the tumor target in vivo

SAg-OX-40 ligand (OX-40L) or 4-1BB ligand (4-1BBL) are fused to a tumor specific targeting structure using recombinant SAgs. A most preferred construct combines the ECD of OX-40L or 4-1BBL with a high affinity tumor specific Fv or fab or (Fab)₂ antibody fragments. The nucleic acids encoding the whole molecule or the ECD of OX-40L (Godfrey et al., supra or 4-1BBL (Goodwin et al., Eur. J. Immunol. 23: 2631-2641 (1993); Melero I. et al., Eur. J. Immunol. 28: 1116-1121 (1998)) are fused in frame with nucleic acids encoding a SAg of any type, although SEG, SEG homologues, egc-SEs, SEP, SEQ, SER, SEU, and Y. pseudotuberculosis are preferred. The SAg may be modified to reduce antigenicity by modifying a dominant epitope and to reduce toxicity by altering its MHC class II binding affinity as described above. The tumor targeting structure may include but is not limited to a tumor receptor ligand or tumor-specific antibody specific or any tumor binding structure listed above under antibody fusion partners or for a fragment thereof. Preferably, the affinity of the tumor targeting structure is of higher affinity than is the affinity of the modified SAg for MHC class II. High affinity antibodies, (fab)₂ or Fab or scFv constructs specific for the OX-40 receptor and 4-1BB receptor that activate costimulatory faction of their target cells to the same degree as OX40L and 4-1BBL may be used in place of the OX40L and 4-1BBL in the SAg-tumor targeting construct.

Such a fusion proteins as described in this section are prepared using a fusion gene comprising nucleic acids encoding the SEG or SEG homologue, the costimulatory molecule and/or the tumor targeting molecule. The vector for recombinant SEG production described herein is useful for this purpose. Additional methodology is described in the art and in Example 2. The same methodology can be used to fuse costimulatory molecules to SEG or an SEG homologue or any useful SAg or SAg homologue described herein. The nucleic acid form of the SAg-costimulatory molecule or SAg-costimulatory-tumor targeting molecule is envisioned as useful.

Spacers commonly used biochemically or recombinantly and bifunctional coupling agents useful for this invention are provided in Forsberg et al., U.S. Pat. No. 7,125,554, issued October 2006; Dohlsten et al., U.S. Pat. No. 6,197,299, issued March 2001; Dohlsten et al., U.S. Pat. No. 5,858,363, issued January 1999 all of which are incorporated by reference in entirety. The spacers best suited to be placed at the N or C terminus of the SEG or other useful SAgs as given above but can be placed in any position that does not interfere with the binding of the SEG, SEG homologue to the TCR or the tumor specific antibody or antibody fragment or tumor ligand to its cognate antigen or receptor.

The SE-OX-40L (or 4-1BB)-tumor targeting fusion protein described above are administered parenterally, intratumorally, intrathecally (e.g., intraperitoneally, intrapleurally) by infusion or injection in conventional or sustained release vehicles in dosages of 0.01 ng/kg to 100 μg/kg using standard protocols or those exemplified herein Frequency of administration may be every 3-7 days. (See sections on Tumor Models and Examples 1 and 2).

### Biochemical Cross-Linkers

In the above fusion polypeptides or conjugates, the SAgs may be linked directly to a fusion partner or fused/conjugated via certain preferred biochemical linker or spacer groups. For chemical conjugates, cross-linking reagents are preferred and are used to form molecular bridges that bond together functional groups of two different molecules. Heterobifunctional crosslinkers can be used to link two different proteins in a step-wise manner while preventing unwanted homopolymer formation. Such cross-linkers are listed in Table 3, below.

Hetero-bifunctional cross-linkers contain two reactive groups one (e.g., N-hydroxy succinimide) generally reacting with primary amine group and the other (e.g., pyridyl disulfide, maleimides, halogens, etc.) reacting with a thiol group. Compositions to be crosslinked therefore generally have, or are derivatized to have, a functional group available. This requirement is not considered to be limiting in that a wide variety of groups can be used in this manner. For example, primary or secondary amine groups, hydrazide or hydrazine. groups, carboxyl, hydroxyl, phosphate, or alkylating groups may be used for binding or cross-linking.

The spacer arm between the two reactive groups of a cross-linker may be of various length and chemical composition. A longer, aliphatic spacer arm allows a more flexible linkage while certain chemical groups (e.g., benzene group) lend extra stability or rigidity to the reactive groups or increased resistance of the chemical link to the action of various agents (e.g., disulfide bond resistant to reducing agents). Peptide spacers, such as Leu-Ala-Leu-Ala, are also contemplated.

It is preferred that a cross-linker have reasonable stability in blood. Numerous known disulfide bond-containing linkers can be used to conjugate two polypeptides. Linkers that contain a disulfide bond that is sterically hindered may give greater stability in vivo, preventing release of the agent prior to binding at the desired site of action.

A most preferred cross-linking reagents for use in with antibody chains is SMPT, a bifunctional cross-linker containing a disulfide bond that is “sterically hindered” by an adjacent benzene ring and methyl groups. Such steric hindrance of the disulfide bond may protect the bond from attack by thiolate anions (e.g., glutathione) which can be present in tissues and blood, and thereby help in preventing decoupling of the conjugate prior to the delivery to the target, preferably tumor, site. SMPT cross-links functional groups such as —SH or primary amines (e.g., the ε-amino group of Lys).

TABLE 3
Hetero-Bifunctional Cross-linkers

| | | Spacer arm length |
|---|---|---|
| Linker | Advantages and Applications | after cross linking |
| Succinimidyloxycarbonyl-α-(2- | Greater stability | 11.2 A |
| pyridyldithio)toluene (SMPT) ¹ | | |
| N-succinimidyl 3-(2- | Thiolation | 6.8 A |
| pyridyldithio)propionate (SPDP) ² | | |
| Sulfosuccinimidyl-6-[α-methyl-α-(2- | Extended spacer arm; | 15.6 A |
| pyridyldithio)toluamido]hexanoate | Water-soluble | |
| (Sulfo-LC-SPDP) ¹ | | |
| Succinimidyl-4-(N- | Stable maleimide reactive group; | 11.6 A |
| maleimidomethyl)cyclohexane-1- | conjugation of enzyme or other | |
| carboxylate (SMCC) ¹ | polypeptide to antibody | |
| Succimimidyl-4-(N- | Stable maleimide reactive group; | 11.6 A |
| maleimidomethyl)cyclohexane- | water-soluble | |
| carboxylate (Sulfo-SMCC) ¹ | | |
| m-Maleimidobenzoyl-N- | Enzyme-antibody conjugation; | 9.9 A |
| hydroxysuccinimide (MBS) ¹ | hapten-carrier protein conjugation | |
| m-Maleimidobenzoyl-N- | Water-soluble | 9.9 A |
| hydroxysulfosuccinimide (Sulfo-MBS) ¹ | | |
| N-Succinimidyl(4- | Enzyme-antibody conjugation | 10.6 A |
| iodacetyl)aminobenzoate (SIAB) ¹ | | |
| Sulfosuccinimidyl(4- | Water-soluble | 10.6 A |
| iodoacetyl)aminobenzoate (Sulfo- | | |
| SIAB) ¹ | | |
| Succinimidyl-4-(p- | Enzyme-antibody conjugation; | 14.5 A |
| maleimidophenyl)butyrate (SMPB) ¹ | extended spacer arm | |
| Sulfosuccinimidyl-4-(p- | Extended spacer arm | 14.5 A |
| maleimidophenyl)butyrate (Sulfo- | Water-soluble | |
| SMPB) ¹ | | |
| 1-ethyl-3-(3-dimethylaminopropyl). | Hapten-Carrier conjugation | 0 |
| carbodiimide hydrochloride (EDC) + | | |
| N-hydroxysulfosuccinimide (sulfoNHS) ³ | | |
| p-Azidobenzoyl hydrazide (ABH) ⁴ | Reacts with sugar groups | 11.9 A |

¹ Reactive toward primary amines, sulfhydryls
² Reactive toward primary amines
³ Reactive toward primary amines. carboxyl groups
⁴ Reactive toward carbohydrates, nonselective

Hetero-bifunctional photoreactive phenylazides containing a cleavable disulfide bond, for example, sulfosuccinimidyl-2-(p-azido salicylamido)-ethyl-1,3′-dithiopropionate. The N-hydroxy-succinimidyl group reacts with primary amino groups and the phenylazide (upon photolysis) reacts non-selectively with any amino acid residue.

Other useful cross-linkers, not considered to contain or generate a protected disulfide, include SATA, SPDP and 2-iminothiolane. The use of such cross-linkers is well known in the art.

Once conjugated, the conjugate is separated from unconjugated SAg and fusion partner polypeptides and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

### Tumors

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

### Chemotherapeutic and Other Agents

Chemotherapeutic agents can be used before, together with or after parenteral/systemic-administration of modified superantigens, fragments, homologues or fusion proteins as described herein. Superantigens are delivered by injection, instillation or infusion by any route including intravenously, intramuscularly, intradermally, intravesicularly, intrathecally, intrapleurally, intrapericardially, subcutaneously, intraperitoneally, and any other parenteral route. Chemotherapy is administered by infusion, instillation or injection by any parenteral route such as intrathecally, intratumorally, intravenously, intratumorally, intramuscularly, intradermally, intravesicularly, intrathecally, intrapleurally, intrapericardially, subcutaneously, intraperitoneally. Preferably chemotherapy is given before, together with, or 1-12 days after superantigen administration. Anti-cancer chemotherapeutic drugs useful in this invention include but are not limited to antimetabolites, anthracycline, vinca alkaloid, anti-tubulin drugs, antibiotics and alkylating agents. Representative specific drugs that can be used alone or in combination include cisplatinum (CDDP), adriamycin, dactinomycin, mitomycin, caminomycin, daunomycin, doxorubicin, tamoxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, aminopterin, combretastatin(s) and derivatives and prodrugs thereof.

A variety of chemotherapeutic and pharmacological agents may be given separately. Those of ordinary skill in the art will know how to select appropriate agents and doses, although, as disclosed, the doses of chemotherapeutic drugs are preferably reduced when used in combination with sickle erythrocyte in the present invention.

Another newer class of drugs that are also termed “chemotherapeutic agents” comprises agents that induce apoptosis. Any one or more of such drugs, including genes, vectors, antisense constructs, siRNA constructs, and ribozymes, as appropriate, may be used in conjunction with the above agents.

Other agents useful herein are anti-angiogenic agents, such as Avastin, angiostatin, endostatin, vasculostatin, canstatin and maspin. Avastin or Bevacizumab is a recombinant humanized monoclonal antibody directed against vascular endothelial growth factor (VEGF). Human VEGF mediates neo-angiogenesis in normal and malignant vasculature. It is overexpressed in most malignancies, and high levels have correlated with a greater risk of metastasis. Avastin or bevacizumab binds VEGF and prevents its interaction with receptors (Flt-1 and KDR) on the surface of endothelial cells. Avastin 5 mg/kg intravenously is given every 14 days until disease progression is detected. The initial dose of Avastin is delivered over 90 minutes as an IV infusion. SS heme is administered before, during or after avastin and usually given once or twice weekly for up to 10 weeks.

Chemotherapeutic agents are administered as single agents or multidrug combinations, in full or reduced dosage per treatment cycle. They can be administered before, during or after sickle erythrocyte composition. In a preferred schedule, the chemotherapeutic agent is administered within 36 hours of the last of two to four treatments of sickle erythrocyte compositions administered intravenously.

The choice of chemotherapeutic drug in such combinations is determined by the nature of the underlying malignancy. For lung tumors, cisplatinum is preferred. For breast cancer, a microtubule inhibitor such as taxotere is the preferred. For malignant ascites due to gastrointestinal tumors, 5-FU is preferred. “Low dose” as used with a chemotherapeutic drug refers to the dose of single agents that is 10-95% below that of the approved dosage for that agent (by the U.S. Food and Drug Administration, FDA). If the regimen consists of combination chemotherapy, then each drug dose is reduced by the same percentage. A reduction of >50% of the FDA approved dosage is preferred although therapeutic effects are seen with dosages above or below this level, with minimal side effects.

The chemotherapeutic agent(s) selected for therapy of a particular tumor preferably is one with the highest response rates against that type of tumor. For example, for non-small cell lung cancer (NSCLC), cisplatinum-based drugs have been proven effective. Cisplatinum may be given parenterally or intratumorally. Other agents useful in NSCLC include the taxanes (paclitaxel and docetaxel), vinca alkaloids (vinorelbine), antimetabolites (gemcitabine), and camptothecin (irinotecan) both as single agents and in combination with a platinum agent.

The optimal chemotherapeutic agents and combined regimens for all the major human tumors are set forth in Bethesda Handbook of Clinical Oncology, Abraham J et al., Lippincott William & Wilkins, Philadelphia, Pa. (2001); Manual of Clinical Oncology, Fourth Edition, Casciato, D A et al., Lippincott William & Wilkins, Philadelphia, Pa. (2000) both of which are herein incorporated in entirety by reference.

Other agents and therapies that are useful before, together with or after parenteral (e.g., intratumoral, intrapleural, intraperitoneal, intravesicular, intravenous) superantigens include, radiotherapeutic agents, antitumor antibodies with attached anti-tumor drugs such as plant-, fungus-, or bacteria-derived toxin or coagulant, ricin A chain, deglycosylated ricin A chain, ribosome inactivating proteins, sarcins, gelonin, aspergillin, restricticin, a ribonuclease, a epipodophyllotoxin, diphtheria toxin, or Pseudomonas exotoxin. Additional cytotoxic, cytostatic or anti-cellular agents capable of killing or suppressing the growth or division of tumor cells include anti-angiogenic agents, interferons alpha and gamma, apoptosis-inducing agents, coagulants, prodrugs or tumor targeted forms, tyrosine kinase inhibitors (Siemeister et al., Cancer Metastasis Rev. 17:241-8 (1998), antisense strategies, RNA aptamers, siRNA and ribozymes against VEGF or VEGF receptors (Saleh M et al., Cancer Res. 56:393-401 (1996); Cheng et al., Proc Natl Acad Sci 93:8502-7 (1996); Ke et al., Int J Oncol. 12:1391-6 (1998); Parry et al., Antisense Nucleic Acid Drug Dev. 9:271-7 (1999)); each incorporated herein by reference.

Any of a number of tyrosine kinase inhibitors is useful when administered before, together with, or after, intratumoral SS heme. These include, for example, the 4-aminopyrrolo[2,3-d]pyrimidines (U.S. Pat. No. 5,639,757). Further examples of small organic molecules capable of modulating tyrosine kinase signal transduction via the VEGF-R2 receptor are the quinazoline compounds and compositions (U.S. Pat. No. 5,792,771). Tarceva or Erlotinib attaches to EGF receptors and thereby blocks the EGF-mediated activation of tyrosine kinase. Tarceva 150 mg daily is administered before during or after parenteral (intrathecal, intrapleural and/or intravenous) sickle erythrocyte treatment and continued until disease progression or unacceptable toxicity occurs.

Other agents which may be employed in combination with superantigens are steroids such as the angiostatic 4,9(11)-steroids and C21-oxygenated steroids (U.S. Pat. No. 5,972,922). Thalidomide and related compounds, precursors, analogs, metabolites and hydrolysis products (U.S. Pat. Nos. 5,712,291 and 5,593,990) may also be used in combination with SAgs and other chemotherapeutic drugs agents to inhibit angiogenesis. These thalidomide and related compounds can be administered orally.

Certain anti-angiogenic agents that cause tumor regression may be administered before, together with, or after, intrathecal, intrapleural, intratumoral, intravenous or parenteral SS heme. These include the bacterial polysaccharide CM101 (currently in clinical trials as an anti-cancer drug) and the antibody LM609. CM101 has been well characterized for its ability to induce neovascular inflammation in tumors. CM101 binds to and cross-links receptors expressed on dedifferentiated endothelium that stimulate the activation of the complement system. It also initiates a cytokine-driven inflammatory response that selectively targets the tumor. CM101 is a uniquely antiangiogenic agent that down-regulates the expression VEGF and its receptors. Thrombospondin (TSP-1) and platelet factor 4 (PF4) may also be used together with or after intratumoral SAg. These are both angiogenesis inhibitors that associate with heparin and are found in platelet α granules.

Interferons and metalloproteinase inhibitors are two other classes of naturally occurring angiogenic inhibitors that can be used before, together with or after SAg administration. Vascular tumors in particular are sensitive to interferon; for example, proliferating hemangiomas are successfully treated with IFNα. Tissue inhibitors of metalloproteinases (TIMPs), a family of naturally occurring inhibitors of matrix metalloproteases (MMPs), can also inhibit angiogenesis and can be used in combination (before, during or after) the SS heme.

### Radiation Therapy

Local radiation to any tumor sites or the mediastinum using the traditional standard dose of 60-65 gy is given concomitant with parenteral (e.g., intrathecal, intravenous, intravesicular, intrapleural, intralymphatic or intratumoral) administration of SAg. The radiotherapy is also be given before, during or after the SAg therapy but in either case there is a hiatus of no more than 30 days between the start of SS heme therapy and the start or conclusion of radiotherapy. The median survival of patients given this type of radiotherapy alone is 5% at one year whereas the combined modality improves the median survival to more than two years.

In general, local radiation therapy alone has minimal efficacy in contributing to long-term disease control in advanced carcinomas. While radiation is an effective palliative measure to relieve symptoms, only a very small minority of patients achieve long-term survival when treated with radiation alone. However, radiation synergizes with SS heme therapy in shrinking tumors and prolonging survival. Radiation is given to bulky or symptomatic lung lesions before, during or after SAg therapy. Preferably it is started 1-2 weeks before the compositions described herein and continued simultaneously with these agents for 1-4 weeks until the full courses of these compositions and radiation are completed. Radiation may also be started after administration of the compositions preferably within 24 hours of the last treatment. Radiation may also be given to a malignant lesion or a tumorous body cavity before, together with or after the site has been injected with the superantigen or heme agents as described herein intratumorally or intrathecally. It may also be administered to a malignant lesion or site not injected specifically with these agents. In this case superantigen agents may be given systemically or intrathecally but not directly to the radiated tumor mass or site. Radiation may also be used together with chemotherapy and systemic and/or intratumoral/intrathecal treatment with the superantigens compositions described herein.

Radiation techniques are preferably continuous rather than split. Hyper-fractionated radiation, employing multiple daily fractions of radiation is preferred to conventionally fractionated radiation. Radiation doses vary from 40-70 gy although a dose between 60 and 70 gy dose is preferred.

### Production and Isolation of Superantigens

The superantigens disclosed herein are prepared by either biochemical isolation, or, preferably by recombinant methods. The following SAgs, including their sequences and biological activities have been known for a number of years. Studies of these SAgs are found throughout the biomedical literature. For, biochemical and recombinant preparation of these SAgs see the following references: Borst D W et al., Infect. Immun. 61: 5421-5425 (1993); Couch J L et al., J. Bacteriol. 170: 2954-2960 (1988); Jones C L et al., J. Bacteriol. 166: 29-33 (1986); Bayles K W et al., Bacteriol. 171: 4799-4806 (1989); Blomster-Hautamaa, D A et al., J. Biol. Chem. 261:15783-15786 (1986); Johnson, L P et al., Mol. Gen. Genet. 203, 354-356 (1986); Bohach G A et al., Infect. Immun. 55: 428-433 (1987); Iandolo J J et al., Meth. Enzymol 165:43-52 (1988); Spero L et al., Meth. Enzymol 78(Pt A):331-6 (1981); Blomster-Hautamaa D A, Meth. Enzymol 165: 37-43 (1988); Iandolo J J Ann. Rev. Microbiol. 43: 375-402 (1989); U.S. Pat. No. 6,126,945 and U.S. provisional patent application 60/389,366 filed Jun. 15, 2002. These references and the references cited therein are hereby incorporated by reference in their entirety.

These SAgs are Staphylococcal enterotoxin A (SEA), Staphylococcal enterotoxin B (SEB), Staphylococcal enterotoxin C (SEC—actually three different proteins, SEC1, SEC2 and SEC3)), Staphylococcal enterotoxin D (SED), Staphylococcal enterotoxin E (SEE) and toxic shock syndrome toxin-1 (TSST-1) (U.S. Pat. No. 6,126,945 and U.S. provisional patent application 60/389,366 filed Jun. 15, 2002, and the references cited therein). The amino acids sequences of the above group of native (wild-type) SAgs are given in the following: SEA (Huang I Y et al., J. Biol. Chem. 262:7006-7013 (1987)); SEB (Papageorgiou A C et al. J. Mol. Biol. 277:61-79 (1998)); SEC1 (Bohach G A et al., Mol. Gen. Genet. 209:15-20 (1987)); SEC2 (Papageorgiou A C et al., Structure 3:769-779 (1995)); SEC3 (Hovde C J et al., Mol. Gen. Genet. 220:329-333 (1990)); SED (Bayles K W et al., J. Bacteriol. 171:4799-4806 (1989)); SEE (Couch J L et al., J. Bacteriol. 170:2954-2960 (1988));TSST-1 (Prasad G S et al., Protein Sci. 6:1220-1227 (1997))

The sections which follow discuss SAgs which have been discovered and characterized more recently.

### Staphylococcal Enterotoxins SEG, SEH, SEL SEJ, SEK, SEL, SEM, SEN, SEQ, SEP, SEQ, SER, SEU

Production of the above staphylococcal enterotoxins are described in full in US patent application PCTUS05/022638 filed Jun. 27, 2005 which is incorporated by reference in entirety.

New Staphylococcal enterotoxins G, H, 1, J, K, L and M (SEG, SEH, SEI, SEJ, SEK, SEL, SEM, SEN, SEQ, SEP, SEQ, SER, SEU; abbreviated below as “SEG-SEU”) were described in Jarraud, S. et al., J. Immunol. 166: 669-677 (2001); Jarraud S et al., J. Clin. Microbiol. 37: 2446-2449 (1999) and Munson, S H et al., Infect. Immun. 66:3337-3345 (1998). SEG-SEU show superantigenic activity and are capable of inducing tumoricidal effects. The homology of these SE's to the better known SE's in the family ranges from 27-64%. Each induces selective expansion of TCR Vβ subsets. Thus, these SEs retain the characteristics of T cell activation and Vβ usage common to all the other SE's. RT-PCR was used to show that SEH stimulates human T cells via the Vα domain of TCR, in particular Vα (TRAV27), while no TCR Vβ-specific expansion was seen. This is in sharp contrast to all other studied bacterial superantigens, which are highly specific for TCR Vβ. Vβ binding superantigens form one group, whereas SEH has different properties that fit well with Vα reactivity. It is suggested that SEH directly interacts with the TCR Vα domain (Petersson K et al., J Immunol. 170:4148-54 (2003)).

SEG and SEH of this group and other enterotoxins including SPEA, SPEC, SPEG, SPEH, SME-Z, SME-Z2, (see below) utilize zinc as part of high affinity MHC class II receptor. Amino acid substitution(s) at the high-affinity, zinc-dependent class II binding site are created to reduce their affinity for MHC class II molecules.

Jarraud S et al., 2001, supra, discloses methods used to identify and characterize egc SEs SEG-SEM, and for cloning and recombinant expression of these proteins. The egc comprises SEG, SEI, SEM, SEN, SEQ and pseudogene products designated ψent 1 and ψent 2. Purified recombinant SEN, SEM, SEI, SEQ, and SEGL29P (a mutant of SEN) were expressed in E. coli. Recombinant SEG, SEN, SEM, SEI, and SEQ consistently induced selective expansion of distinct subpopulations of T cells expressing particular Vβ genes.

Jarraud S et al., 2001, supra, indicates that the seven genes and pseudogenes composing the egc (enterotoxin gene cluster) operon are co-transcribed. The association of related co-transcribed genes suggested that the resulting peptides might have complementary effects on the host's immune response. One hypothesis is that gene recombination created new SE variants differing by their superantigen activity profiles. By contrast, SEGL29P failed to trigger expansion of any of 23 Vβ subsets, and the L29P mutation accounted for the complete loss of superantigen activity (although this mutation did not induce a major conformational change). It is believed that this substitution mutation located at a position crucial for proper superantigen/MHC II interaction.

Overall, TCR repertoire analysis confirms the superantigenic nature of SEG, SEI, SEM, SEN, SEQ. These investigators used a number of TCR-specific mAbs (Vβ specificity indicated in brackets) for flow cytometric analysis: E2.2E7.2 (Vβ2), LE89 (Vβ3), IMMU157 (Vβ5.1), 3D11 (Vβ5.3), CRI304.3 (Vβ6.2), 3G5D15 (Vβ7), 56C5.2 (Vβ8.1/8.2), FIN9 (Vβ9), C21 (Vβ11), S511 (Vβ12), IMMU1222 (Vβ13.1), JIJ74 (Vβ13.6), CAS1.1.13 (Vβ14), Tamaya1.2 (Vβ16), E17.5F3 (Vβ17), βA62.6 (Vβ18), ELL1.4 (Vβ20), IG125 (Vβ21.3), IMMU546 (Vβ22), and HUT78.1 (Vβ23). Flow cytometry also revealed preferential expansion of CD4+ T cells in SEI and SEM cultures. By contrast, the CD4/CD8 ratios in SEQ-, SEN-, and SEG-stimulated T cell lines were close to those in fresh PBL.

Recombinant and biochemical preparation of the egc SEs is given in U.S. 60/799,514, PCTUS05/022638, US60/583,692, US60/665,654, US60/626,159 which are incorporated by reference and their references in their entirety.

Our most current methodology for manufacture of SEG and SEGₗₑᵤ47_{arg} yielding up to 300 mg of egc-SE's and SEGₗₑᵤ47_{arg} homologue with 98% purity is given as follows.

The prokaryotic expression cassette for the SEG was codon optimized and built synthetically and the gene was cloned into the pET24b(+) expression plasmid (kanamycin resistant) at the NdeI restriction site to avoid the addition of any tags onto the protein.

Following the gene sequence, two STOP codons were inserted to prevent any read-through onto the His tag sequence present on the 3′ end of the MCS in the pET24b(+) vector. Signal sequences utilized by Staphylococcus aureus for protein activation and posttranslational shuttling were excluded leaving only the amino acid sequence of the mature peptide. The lyophilized DNA was suspended in 10 mM Tris/1 mM EDTA (pH 8) in a Class 100 BSC and then aliquoted on dead reckoning at 200 ng/vial (20 ng/μl). The vials were frozen at −80° C. and entered into the clinical management and storage system within the BSL2 laboratory.

### Growth and Cell Lysis

1. The pET24b-SEG is transformed into BL21 (DE3) Veggie™ and expressed using an auto-induction medium (TBII derivative containing 0.4% lactose). The culture is grown for 20 hours at 30° C., 200 rpm, resulting in ˜20 g/L wet weight biomass (harvested by centrifugation).
2. The cells are resuspended in a solution containing 50 mM Tris-HCl, 5 mM EDTA, 10 mM BME, and 1% Triton X-100. The cell suspension is sonicated using a Branson Sonifier at a 50% Duty Cycle and an Output Power of 4 for a total sonication time of 1 min/gram.
3. The lysate is clarified by centrifugation at 15,000×g for 30 minutes. The resulting pellets are resuspended in the same solution and treated to a second round of sonication and clarification.
4. The lysates from each round of sonication are pooled prior to the first chromatography step (approx. 1500 mg of soluble protein is extracted per liter of culture)

### Chromatography and Buffer Exchange

1. The clarified lysate is loaded onto a Q/SP Sepharose (mixed bed ion exchange) column and the load flow is collected for subsequent purification.
2. The load flow through from the Q/SP chromatography is diluted with a 50 mM MES, pH 5.5 buffer, 0.45 gm filtered, and loaded onto a SP Sepharose column. A gradient is run from 0-300 mM NaCl in 50 mM MES, pH 5.5 and fractions are collected, neutralized with Tris, and analyzed with SDS-PAGE.
3. Selected fractions from the CEX capture are pooled for further purification. The pooled post-CEX capture solution is diluted with an equal volume of 4.0 M (NH4)2SO4, 50 mM Tris, pH 8.0, 0.45 μm filtered, and loaded onto an Octyl Sepharose Fast Flow column. A gradient is run from 2.0-1.0 M (NH4)2SO4 and fractions are collected. Samples of each fraction are buffer exchanged and analyzed with SDS-PAGE.
4. Selected fractions from the HIC capture are pooled for further purification. The pooled fractions are diafiltered into 50 mM Tris, pH 7.0 on a 5 kDa Minimate system. The concentrated and buffer exchanged SEG is then loaded over a Q Sepharose Fast Flow column and the load flow is collected.
5. The LFT from the AEX void chromatography step is then ultrafiltered on a 5 kDa Minimate system for volume reduction prior to gel filtration.
6. The retentate from the ultrafiltration is 0.45 μm filtered and then loaded onto a Sephacryl S-200 HR gel filtration column equilibrated with 1×PBS, pH 7.4.
7. All peaks are collected in fractions and analyzed with SDS-PAGE and silver staining. Selected fractions are pooled, 0.22 μm filtered, and samples transferred to Quality Control for analysis.

The references to amino acid sequences of SEG-SEU are incorporated by reference and their references in entirety as follows: SEG (Baba, T. et al., Lancet 359, 1819-1827 (2002)); SEG (Jarraud, S et al., J. Immunol. 166: 669-677 (2001)); SEH (Omoe, K. et al., J. Clin. Microbiol. 40: 857-862 (2002));SEI (Kuroda, M. et al., Lancet 357 (9264), 1225-1240 (2001));SEJ (Zhang S. et al., FEMS Microbiol. Lett. 168:227-233 (1998)); SEK (Baba T., et al., Lancet 359: 1819-1827 (2002)); SEL (Kuroda M. et al., Lancet 357: 1225-1240 (2001)); SEM (Kuroda M. et al., Lancet 357: 1225-1240 (2001)); SEN (Jarraud S et al., J. Immunol. 166: 669-677 (2001)); SEO (Jarraud S et al., J. Immunol. 166: 669-677 (2001)); ψent 1 (Jarraud S et al., J. Immunol. 166: 669-677 (2001)); ψent 2 (Jarraud S et al., J. Immunol. 166: 669-677 (2001)); SEP (Kuroda M. et al., Lancet 357, 1225-1240 (2001); SEQ (Lindsay, J A et al., Mol. Microbiol. 29, 527-543 (1998)); SER Omoe K et al., ACCESSION BAC97795; SEU (Letertre C et al., J. Appl. Microbiol. 95, 38-43 (2003)).

### Functional Fragments, Homologues and Derivatives of Superantigens and Therapeutic Proteins Described Herein

The present invention contemplates, the use of homologues of wild type proteins such as superantigens that have the requisite biological activity to be useful in accordance with the invention.

Thus, in addition to native proteins and nucleic acid compositions described herein, the present invention encompasses functional derivatives, among which homologues are preferred. By “functional derivative” is meant a “fragment,” “variant,” “mutant,” “homologue,” “analogue,” or “chemical derivative. Homologues include fusion proteins, chimeric proteins and conjugates that include a SAg portion fused to or conjugated to a fusion partner polypeptide or peptide. A functional derivative retains at least a portion of the biological activity of the native protein which permits its utility in accordance with the present invention. For superantigens, such biological activity includes stimulation of T cell proliferation and/or cytokine secretion, stimulation of T cell-mediated cytotoxic activity, as a result of interactions of the SAg composition with T cells preferably via the TCR Vβ or Vα region.

A “fragment” refers to any shorter peptide. A “variant” refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from the same or a different species. Homologues, as used herein, typically share at least about 50% sequence similarity at the DNA level or at least about 18% sequence similarity at the amino acid level, with a native protein.

An “analogue” refers to a non-natural molecule substantially similar to either the entire molecule or a fragment thereof.

A “chemical derivative” contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

A fusion protein comprises a native protein, a fragment or a homologue fused by recombinant means to another polypeptide fusion partner, optionally including a spacer between the two sequences. Preferred fusion partners are antibodies, Fab fragments, single chain Fv fragments. Other fusion partners are any peptidic receptor, ligand, cytokine, domain (“ECD”) of a molecule and the like.

The recognition that the biologically active regions of the proteins, for example, are substantially homologous, i.e., that the sequences are substantially similar, enables prediction of the sequences of synthetic peptides which will exhibit similar biological effects in accordance with this invention.

The following terms are used in the disclosure of sequences and sequence relationships between two or more nucleic acids or polypeptides: (a) “reference sequence”, (b) “comparison window”, (c) “sequence identity”, (d) “percentage of sequence identity”, and (e) “substantial identity”

As used herein, “reference sequence” is a defined sequence used as a basis for sequence comparison. A reference sequence may be a subset or the entirety of a specified sequence; for example, as a segment of a full-length cDNA or other polynucleotide sequence, or the complete cDNA or polynucleotide sequence. The same is the case for polypeptides and their amino acid sequences.

As used herein, “comparison window” includes reference to a contiguous and specified segment of a polynucleotide or amino acid sequence, wherein the sequence may be compared to a reference sequence and wherein the portion of the sequence in the comparison window may comprise additions or deletions (i.e., gaps) compared to the reference sequence (which does not comprise additions or deletions) for optimal alignment of the two sequences.

Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of nucleotide and amino acid sequences for comparison are well-known in the art. For comparison, optimal alignment of sequences may be done using any suitable algorithm, of which the following are examples:
(a) the local homology algorithm (“Best Fit”) of Smith and Waterman, Adv. Appl. Math. 2: 482 (1981);
(b) the homology alignment algorithm (GAP) of Needleman and Wunsch, J. Mol. Biol. 48: 443 (1970); or
(c) a search for similarity method (FASTA and TFASTA) of Pearson and Lipman, Proc. Natl. Acad. Sci. 85 2444 (1988);

In a preferred method of alignment, Cys residues are aligned. Computerized implementations of these algorithms, include, but are not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (Madison, Wis.). The CLUSTAL program is described by Higgins et al., Gene 73:237-244 (1988); Higgins et al., CABIOS 5:151-153 (1989); Corpet et al., Nuc Acids Res 16:881-90 (1988); Huang et al., CABIOS 8:155-65 (1992), and Pearson et al., Methods in Molecular Biology 24:307-331 (1994).

A preferred program for optimal global alignment of multiple sequences is PileUp (Feng and Doolittle, J Mol Evol 25:351-360 (1987) which is similar to the method described by Higgins et al., 1989, supra).

The BLAST family of programs which can be used for database similarity searches includes: NBLAST for nucleotide query sequences against database nucleotide sequences; XBLAST for nucleotide query sequences against database protein sequences; BLASTP for protein query sequences against database protein sequences; TBLASTN for protein query sequences against database nucleotide sequences; and TBLASTX for nucleotide query sequences against database nucleotide sequences. See, for example, Ausubel et al., eds., Current Protocols in Molecular Biology, Chapter 19, Greene Publishing and Wiley-Interscience, New York (1995) or most recent edition. Unless otherwise stated, stated sequence identity/similarity values provided herein, typically in percentages, are derived using the BLAST 2.0 suite of programs (or updates thereof) using default parameters. Altschul et al., Nuc Acids Res. 25:3389-3402 (1997).

As is known in the art, BLAST searches assume that proteins can be modeled as random sequences. However, many real proteins comprise regions of nonrandom sequence which may include homopolymeric tracts, short-period repeats, or regions rich in particular amino acids. Alignment of such regions of “low-complexity” regions between unrelated proteins may be performed even though other regions are entirely dissimilar. A number of low-complexity filter programs are known that reduce such low-complexity alignments. For example, the SEG (Wooten et al., Comput. Chem. 17:149-163 (1993) and XNU (Claverie et al., Comput. Chem., 17:191-201 (1993) low-complexity filters can be employed alone or in combination.

As used herein, “sequence identity” or “identity” in the context of two nucleic acid or amino acid sequences refers to the residues in the two sequences which are the same when aligned for maximum correspondence over a specified comparison window. It is recognized that when using percentages of sequence identity for proteins, a residue position which is not identical often differs by a conservative amino acid substitution, where a substituting residue has similar chemical properties (e.g., charge, hydrophobicity, etc.) and therefore does not change the functional properties of the polypeptide. Where sequences differ in conservative substitutions, the % sequence identity may be adjusted upwards to correct for the conservative nature of the substitution, and be expressed as “sequence similarity” or “similarity” (combination of identity and differences that are conservative substitutions). Means for making this adjustment are well-known in the art. Typically this involves scoring a conservative substitution as a partial rather than as a full mismatch, thereby increasing the percentage sequence identity. Thus, for example, where an identical amino acid is given a score of “1” and a non-conservative substitution is given a score of “0” zero, a conservative substitution is given a score between 0 and 1. The scoring of conservative substitutions is calculated, e.g., according to the algorithm of Meyers et al., CABIOS 4:11-17 (1988) as implemented in the program PC/GENE (Intelligenetics, Mountain View, Calif., USA). As used herein, “percentage of sequence identity” refers to a value determined by comparing two optimally aligned sequences over a comparison window, wherein the portion of the nucleotide or amino acid sequence in the comparison window may comprise additions or deletions (i.e., gaps) as compared to the reference sequence (which lacks such additions or deletions) for optimal alignment, such as by the GAP algorithm (supra). The percentage is calculated by determining the number of positions at which the identical nucleotide or amino acid residue occurs in both sequences to yield the number of matched positions, dividing that number by the total number of positions in the window of comparison and multiplying the result by 100, thereby calculating the percentage of sequence identity.

The term “substantial identity” of two sequences means that a polynucleotide or polypeptide comprises a sequence that has at least 60%, preferably at least 70%, more preferably at least 80%, even more preferably at least 90%, and most preferably at least 95% sequence identity to a reference sequence using one of the alignment programs described herein using standard parameters. Values can be appropriately adjusted to determine corresponding identity of the proteins encoded by two nucleotide sequences by taking into account codon degeneracy, amino acid similarity, reading frame positioning, etc.

One indication that two nucleotide sequences are substantially identical is if they hybridize to one other under stringent conditions. Because of the degeneracy of the genetic code, a number of different nucleotide codons may encode the same amino acid. Hence, two given DNA sequences could encode the same polypeptide but not hybridize under stringent conditions. Another indication that two nucleic acid sequences are substantially identical is that the polypeptide encoded by the first nucleic acid is immunologically cross reactive with the polypeptide encoded by the second nucleic acid. Clearly, then, two peptide or polypeptide sequences are substantially identical if one is immunologically reactive with antibodies raised against the other. A first peptide is substantially identical to a second peptide, if they differ only by a conservative substitution. Peptides which are “substantially similar” share sequences as noted above except that nonidentical residue positions may differ by conservative substitutions.

Thus, in one embodiment of the present invention, the Lipman-Pearson FASTA or FASTP program packages (Pearson, W. R. et. al., 1988, supra; Lipman, D. J. et al, Science 227:1435-1441 (1985)) in any of its older or newer iterations may be used to determine sequence identity or homology of a given protein, preferably using the BLOSUM 50 or PAM 250 scoring matrix, gap penalties of −12 and −2 and the PIR or SwissPROT databases for comparison and analysis purposes. The results are expressed as z values or E ( ) values. To achieve a more “updated” z value cutoff for statistical significance, preferably corresponding to a z value >10 based on the increase in database size over that of 1988, in a FASTA analysis using the equivalent 2001 database, a significant z value would exceed 13.

A more widely used and preferred methodology determines the Percent identity of two amino acid sequences or of two nucleic acid sequences after optimal alignment as discussed above, e.g., using BLAST. In a preferred embodiment of this approach, a polypeptide being analyzed for its homology with native protein is at least 20%, preferably at least 40%, more preferably at least 50%, even more preferably at least 60%, and even more preferably at least 70%, 80%, or 90% as long as the reference sequence. The amino acid residues (or nucleotides) at corresponding positions are then compared. Amino acid or nucleic acid “identity” is equivalent to amino acid or nucleic acid “homology”.

In a preferred comparison of a putative polypeptide or peptide homologue polypeptide and a native protein, the percent identity between two amino acid sequences is determined using the Needleman and Wunsch alignment algorithm (incorporated into the GAP program in the GCG software package (available at the URL www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between the encoding nucleotide sequences is determined using the GAP program in the GCG software package (also available at above URL), using a NWSgapdna.CMP matrix and a gap weight of 40, 50, 60, 70, or 80 and a length weight of 1, 2, 3, 4, 5, or 6. In another embodiment, the algorithm of Meyers et al., supra (incorporated into the ALIGN program, version 2.0), is implemented using a PAM120 weight residue table, a gap length penalty of 12 and a gap penalty of 4.

The wild-type (or native) SAg-encoding nucleic acid sequence or the SAg protein sequence can further be used as a “query sequence” to search against a public database, for example, to identify other family members or related sequences. Such searches can be performed using the NBLAST and XBLAST programs, supra (see Altschul et al. (1990) J. Mol. Biol. 215:403-10). BLAST nucleotide searches can be performed with the NBLAST program, score=100, wordlength=12 to identify nucleotide sequences homologous to native SAgs. BLAST protein searches can be performed with the XBLAST program, score=50, wordlength=3 to identify amino acid sequences homologous to identify polypeptide molecules homologous to a native SAg. To obtain gapped alignments for comparison purposes, Gapped BLAST can be utilized as described in Altschul et al. (1997, supra). Default parameters of XBLAST and NBLAST can be found at the NCBI website (www.ncbi.nlm.nih.gov)

Using the FASTA programs and method of Pearson and Lipman, a preferred SAg homologue is one that has a z value >10. Expressed in terms of sequence identity or similarity, a preferred SAg homologue for use according the present invention has at least about 20% identity or 25% similarity to native SAg. Preferred identity or similarity is higher. More preferably, the amino acid sequence of a homologue is substantially identical or substantially similar to a native protein molecule as those terms are defined above.

One group of substitution variants (also homologues) are those in which at least one amino acid residue in the peptide molecule, and preferably, only one, has been removed and a different residue inserted in its place. Deletion and addition variants are also homologues if they satisfy the structural and functional criteria set forth herein with respect to their parent or native molecules. For a detailed description of protein chemistry and structure, see Schulz, G. E. Principles of Protein Structure Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, kg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

More substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, Ile, Phe, Val or Ala; (c) substitution of a Cys residue for (or by) any other residue; (d) substitution of a residue having an electropositive side chain, e.g., Lys, Arg or His, for (or by) a residue having an electronegative charge, e.g., Glu or Asp; or (e) substitution of a residue having a bulky side chain, e.g., Phe, for (or by) a residue not having such a side chain, e.g., Gly.

The deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay of cytotoxicity or biological assay of T cell function as described herein. For non-superantigen homologues, the screening test(s) selected to assay function reflect the intrinsic functional activity of the native protein particularly its tumoricidal activity in the context of the inventions described herein. Modifications of such proteins or peptide properties as redox or thermal stability, hydrophobicity, susceptibility to proteolytic degradation or the tendency to aggregate with carriers or into multimers are assessed by methods well known to the ordinarily skilled artisan.

### Chemical Derivatives

Covalent modifications of the SAg proteins or peptide fragments thereof, preferably of SEs or peptide fragments thereof, are included herein. Such modifications may be introduced into the molecule by reacting targeted amino acid residues of the protein or peptide with an organic derivatizing agent that is capable of reacting with selected side chains or terminal residues. This may be accomplished before or after polymerization.

Cysteinyl residues most commonly are reacted with a-haloacetates (and corresponding amines), such as 2-chloroacetic acid or chloroacetamide, to give carboxymethyl or carboxyamidomethyl derivatives. Cysteinyl residues also are derivatized by reaction with bromotrifluoroacetone, α-bromo-(5-imidozoyl)propionic acid, chloroacetyl phosphate, N-alkylmaleimides, 3-nitro-2-pyridyldisulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate; pyridoxal phosphate; pyridoxal; chloroborohydride; trinitrobenzenesulfonic acid; 0-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimides as noted above. Aspartyl and glutamyl residues are converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein and the like. Moieties capable of mediating such effects are disclosed, for example, in Remington's Pharmaceutical Sciences, 16th ed., Mack Publishing Co., Easton, Pa. (1980).

### Superantigen Homologues

The variants or homologues of native SAg proteins or peptides including mutants

(substitution, deletion and addition types), fusion proteins (or conjugates) with other polypeptides, are characterized by substantial sequence homology to
(a) the long-known SE's—SEA, SEB, SEC1-3, SED, SEE and TSST-1;
(b) long-known SpE's;
(c) more recently discovered SE's (SEG, SEH, SEI, SEJ, SEK, SEL, SEM, SEN, SEO, SEP, SER, SEU, SETs 1-5); or
(d) non-enterotoxin superantigens (YPM, M. arthritides superantigen).
   Preferred homologues were disclosed above.

Table 1 in PCT US05/022638 filed Jun. 27, 2005 incorporated in its entirety by reference lists a number of native SEs and exemplary homologues (amino acid substitution, deletion and addition variants (mutants) and fragments) with z values >10 (range: z=16 to z=136) using the Lipman-Pearson algorithm and FASTA. These homologues also induce significant T lymphocyte mitogenic responses that are generally comparable to native SE's.

In addition, as shown in Table 2 of PCT US05/022638 filed Jun. 27, 2005 incorporated in its entirety by reference, several of these homologues also promote antigen-nonspecific T lymphocyte killing in vitro by a mechanism termed “superantigen-dependent cellular cytotoxicity” (SDCC) or, in the case of SAg-mAb fusion proteins, “superantigen/antibody dependent cellular cytotoxicity (SADCC).”

According to the present invention, other SE homologues (e.g., z>10 or, in another embodiment, having at least about 20% sequence identity or at least about 25% sequence similarity when compared to native SEs), exhibiting T lymphocyte mitogenicity, SDCC or SADCC, are useful anti-tumor agents when administered to a tumor bearing host.

### Pharmaceutical Administration of Heme and Heme Conjugates

Superantigen conjugates and fusion proteins may be administered parenterally preferably intravenously by infusion or injection but also may be injected intratumorally, intrapleurally, intraperitoneally, intrathecally, intrapericardially, intravesicularly, subcutaneously, intralymphatically, intraarticularly, intradermally, intracranially, intraarticularly or intramuscularly. They may be administered in a controlled release formulation.

The pharmaceutical compositions of the present invention will generally comprise an effective amount superantigen conjugate. The SAg conjugates are dissolved or dispersed in a pharmaceutically acceptable carrier or aqueous medium. One or more administrations may be employed, depending upon the lifetime of the drug at the tumor site and the response of the tumor to the drug. Administration may be by syringe, catheter or other convenient means allowing for introduction of a flowable composition. Dosages in mice range from 0.1 ng to 100 ug and in humans from 1 ug to 100 mg. Administration may be every 2-3 days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

Superantigen conjugates and fusion proteins of the present invention are preferably formulated for parenteral administration, e.g., introduction by injection, infusion. They may also be administered intravenously, intramuscularly, intradermally, intraperitoneally, intrapleurally, intraarticularly. Means for preparing aqueous compositions that contain the heme or heme conjugate compositions are known to those of skill in the art in light of the present disclosure. Typically, such compositions can be prepared either as liquid solutions or suspensions; solid forms suitable for using to prepare solutions or suspensions upon the addition of a liquid prior to injection can also be prepared.

The techniques of preparation are generally well known in the art as exemplified by Remington's Pharmaceutical Sciences, 16th Ed. Mack Publishing Company, 1980, or most recent edition, incorporated herein by reference. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

### Pharmaceutical Administration of Superantigens, Modified Superantigens, Superantigen Homologues and Fusion Proteins

One or a plurality of any SAg, SAg homologues, fragments, mutants, fusion proteins and conjugates (SAg agents) or mixtures thereof are administered by injection, infusion, instillation or implantation. Preferably, neutralizing antibodies against the selected SAg to be used are not present in the sera of patients.

The SAg agents may be administered parenterally preferably intravenously by infusion or injection but also may be implanted or injected intratumorally or subcutaneously, intradermally or intramuscularly. They may be administered in a controlled release formulation. SAg agents may be administered intrathecally in patients with malignant intrathecal fluid accumulation due to primary or metastatic tumors, e.g., malignant pleural effusions in patients with lung cancer or metastatic breast, gastric or ovarian cancer. SAg agents may also be administered intrathecally to patients with intrathecal and parenchymal tumor (e.g., involvement of pleura and lung parenchyma) but little or no fluid accumulation in the cavitary space. SAg agents may also be administered intrathecally to patients without malignant involvement or fluid accumulation in the cavitary space or its membranes but with. primary or metastatic tumor of the organ (e.g., lung, stomach) and/or lymph nodes. For example, SAg agents may be administered intrapleurally to patients with primary lung cancer or lung metastases from other primary tumors (e.g., breast, ovary, gastric) without malignant involvement of the pleura or pleural space. In each of the above examples, intrathecal administration of the SAg agents may be administered simultaneously or sequentially with one or a plurality of the SAgs administered intratumorally, intralymphatically or intravenously.

SAg agents are administered every 3-10 days for up to three months. Dosages of individual SAg agents used for intrathecal, intratumoral, intralymphatic and intravenous administration range from 0.1 pg-500 ng.

SAg agents are also administered intratumorally to stimulate a T cell-based inflammatory response, including release of tumoricidal cytokines and induction of cytotoxic T cells. The amount of SAg agents administered to a single tumor site ranges from about 0.05-1 ng/kg body weight. The intratumoral dose of a cytotoxic drug administered to the tumor site will generally range from about 0.1 to 500, more usually about 0.5 to 300 mg/kg body weight, depending upon the nature of the drug, size of tumor, and other considerations.

The pharmaceutical compositions of the present invention will generally comprise an effective amount of at least a SAg composition dissolved or dispersed in a pharmaceutically acceptable carrier or aqueous medium. Combined therapeutics are also contemplated, and the same type of underlying pharmaceutical compositions may be employed for both single and combined medicaments. The intratumoral composition can be administered to the tumor by needle or catheter via percutaneous entry or via endoscopy, bronchoscopy, culdoscopy or other modes of direct vision including directly at the time of surgery. The composition can be localized into the tumor with CT and/or ultrasound guidance.

With each drug in each tumor, experience will provide an optimum level. One or more administrations may be employed, depending upon the lifetime of the drug at the tumor site and the response of the tumor to the drug. Administration may be by syringe, catheter or other convenient means allowing for introduction of a flowable composition into the tumor. Administration may be every three days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

### Tumor Models and Procedures for Evaluating Anti-Tumor Effects Studies

The various superantigen compositions fragments, homologues and fusion proteins in free form are tested for therapeutic efficacy in several well established rodent models which are considered to be highly representative of a broad spectrum of human tumors. These approaches are described in detail in Geran, R. I. et al., “Protocols for Screening Chemical Agents and Natural Products against Animal Tumors and Other Biological Systems (Third Edition)”, Canc. Chemother. Reports, Pt 3, 3:1-112, which is hereby incorporated by reference in its entirety.

### A. Calculation of Mean Survival Time (MST)

MST (days) is calculated according to the formula:

S+AS(A−1)−(B+1)NT/S(A−1)−NT
Day: Day on which deaths are no longer considered due to drug toxicity. For example, with treatment starting on Day 1 for survival systems (such as L1210, P388, B16, 3LL, and W256): Day A=Day 6; Day B=Day beyond which control group survivors are considered “no-takes.”
S: If there are “no-takes” in the treated group, S is the sum from Day A through Day B. If there are no “no-takes” in the treated group, S is the sum of daily survivors from Day A onward.
S(A−1): Number of survivors at the end of Day (A−1).
Example: for 3LE21, S(A−1)=number of survivors on Day 5.
NT: Number of “no-takes” according to the criteria given in Protocols 7.300 and 11.103.

### B. T/C Computed for all Treated Groups

T/C=MST of treated group/MST of control group×100

Treated group animals surviving beyond Day Bare eliminated from calculations (as follows):

| No. of survivors in treated | Percent of “no-takes” | |
|---|---|---|
| group beyond Day B | in control group | Conclusion |
| 1 | Any percent | “no-take” |
| 2 | <10 | drug inhibition |
| | ³10 | “no-takes” |
| ³3 | <15 | drug inhibitions |
| | ³15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

Calculation of Median Survival Time (MedST)

MedST is the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of MedST from Survivors

If the total number of animals including survivors (N) is even, the MedST (days) (X+Y)/2, where X is the earlier day when the number of survivors is N/2, and Y is the earliest day when the number of survivors (N/2)−1. If N is odd, the MedST (days) is X.

D. Computation of MedST from Mortality Distribution

If the total number of animals including survivors (N) is even, the MedST (days) (X+Y)/2, where X is the earliest day when the cumulative number of deaths is N/2, and Y is the earliest day when the cumulative number of deaths is (N/2)+1. If N is odd, the MedST (days) is X. “Cures” and “no-takes” in systems evaluated by MedST are based upon the day of evaluation. On the day of evaluation any survivor not considered a “no-take” is recorded as a “cure.” Survivors on day of evaluation are recorded as “cures” or “no-takes,” but not eliminated from the calculation.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times ( width [ mm ] ) 2}{2} or \frac{L \times ( W ) 2}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of small | Percent of | |
|---|---|---|
| tumors in | “no-takes” in | |
| treated group | control group | Action |
| ≦17 | Any percent | no-take; not used in calculations |
| 18-39 | <10 | drug inhibition; use in calculations |
| | ≧10 | no-takes; not used in calculations |
| ≧40 | <15 | drug inhibition; use in calculations |
| | ≧15 | Code all nontoxic tests “33” |

Positive control compounds are not considered to have “no-takes” regardless of the number of “no-takes” in the control group. Thus, the tumor weights of all surviving animals are used in the calculation of T/C for the positive control (T/C defined above) SDs of the mean control tumor weight are computed the factors in a table designed to estimate SD using the estimating factor for SD given the range (difference between highest and lowest observation) (Biometrik Tables for Statisticians Pearson E S & Hartley H G eds. Cambridge Press, vol. 1, table 22, p. 165).

### II. Specific Tumor Models

### A. Lymphoid Leukemia L1210

Summary: Ascitic fluid from donor mouse is transferred into recipient BDF1 or CDF1 mice. Treatment begins 24 hours after implant. Results are expressed as a percentage of control survival time. The key parameter is mean survival time. Origin of tumor line: induced in 1948 in spleen and lymph nodes of mice by painting skin with MCA (J Natl Cancer Inst. 13:1328 (1953)).

| | |
|---|---|
| Animals | One sex used for all test and control animals in one experiment. |
| Tumor Transfer | Inject ip, 0.1 ml of diluted ascitic fluid containing 10⁵ cells |
| Propagation | DBA/2 mice (or BDF1 or CDF1 for one generation). |
| Time of Transfer | Day 6 or 7 |
| Testing | BDF1 (C57BL/6 × DBA/2) or CDF1 (BALB/c × DBA/2) |
| Time of Transfer | Day 6 or 7 |
| Weight | Within a 3-g range, minimum weight of 18 g for males and 17 g for females. |
| Exp Size (n) | 6/group; No. of control groups varies according to number of test groups. |

### Testing Schedule

| DAY | PROCEDURE |
|---|---|
| 0 | Implant tumor. Prepare materials. Run positive control in every odd-numbered |
| | experiment. Record survivors daily. |
| 1 | Weigh and randomize animals. Begin treatment with therapeutic composition. Typically, |
| | mice receive 1 μg of the test composition in 0.5 ml saline. Controls receive saline alone. |
| | Treatment is one dose/week. Any surviving mice are sacrificed after 4 wks of therapy. |
| 5 | Weigh animals and record. |
| 20 | If there are no survivors except those treated with positive control compound, evaluate |
| 30 | Kill all survivors and evaluate experiment. |

Quality Control: Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%.

Evaluation: Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

### B. Lymphocytic Leukemia P388

Summary: Ascitic fluid from donor mouse is implanted in recipient BDF1 or CDF1 mice. Treatment begins 24 hours after implant. Results are expressed as a percentage of control survival time. The key parameter is MedST. Origin of tumor line: induced in 1955 in a DBA/2 mouse by painting with MCA (Scientific Proceedings, Pathologists and Bacteriologists 33:603 (1957)).

| | |
|---|---|
| Animals | One sex used for all test and control animals in one experiment. |
| Tumor Transfer | Inject ip, 0.1 ml of diluted ascitic fluid containing 10⁶ cells |
| Propagation | DBA/2 mice (or BDF1 or CDF1 for one generation). |
| Time of Transfer | Day 7 |
| Testing | BDF1 (C57BL/6 × DBA/2) or CDF1 (BALB/c × DBA/2) |
| Time of Transfer | Day 6 or 7 |
| Weight | Within a 3-g range, minimum weight of 18 g for males and 17 g for females. |
| Exp Size (n) | 6/group; No. of control groups varies according to number of test groups. |

### Testing Schedule

| DAY | PROCEDURE |
|---|---|
| 0 | Implant tumor. Prepare materials. Run positive control in every odd-numbered |
| | experiment. Record survivors daily. |
| 1 | Weigh and randomize animals. Begin treatment with therapeutic composition. Typically, |
| | mice receive 1 μg of the test composition in 0.5 ml saline. Controls receive saline alone. |
| | Treatment is one dose/week. Any surviving mice are sacrificed after 4 wks of therapy. |
| 5 | Weigh animals and record. |
| 20 | If there are no survivors except those treated with positive control compound, evaluate |
| 30 | Kill all survivors and evaluate experiment. |

Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

Quality Control Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135%. Check control deaths, no takes, etc.

Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

### C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The key parameter is mean survival time. Origin of tumor line: arose spontaneously in 1954 on the skin at the base of the ear in a C57BL/6 mouse (Handbook on Genetically Standardized Jax Mice. Jackson Memorial Laboratory, Bar Harbor, Me., 1962. See also Ann NY Acad Sci 100, Parts 1 and 2, (1963)).

| | |
|---|---|
| Animals | One sex used for all test and control animals in one experiment. |
| Propagation Strain | C57BL/6 mice |
| Tumor Transfer | Implant fragment sc by trochar or 12-g needle or tumor homogenate* |
| | every 10-14 days into axillary region with puncture in inguinal region. |
| Testing Strain | BDF1 (C57BL/6 × DBA/2) |
| Time of Transfer | Excise sc tumor on Day 10-14 from donor mice and implant as above |
| Weight | Within a 3-g range, minimum weight of 18 g for males and 17 g for females. |
| Exp Size (n) | 10/group; No. of control groups varies according to number of test groups. |

*Tumor homogenate: Mix 1 g or tumor with 10 ml of cold balanced salt solution, homogenize, and implant 0.5 ml of tumor homogenate ip or sc. Fragment: A 25-mg fragment may be implanted sc.

### Testing Schedule

| DAY | PROCEDURE |
|---|---|
| 0 | Implant tumor. Prepare materials. Run positive control in every odd-numbered |
| | experiment. Record survivors daily. |
| 1 | Weigh and randomize animals. Begin treatment with therapeutic composition. Typically, |
| | mice receive 1 μg of the test composition in 0.5 ml saline. Controls receive saline alone. |
| | Treatment is one dose/week. Any surviving mice are sacrificed after 8 wks of therapy. |
| 5 | Weigh animals and record. |
| 60 | Kill all survivors and evaluate experiment. |

Quality Control: Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. Mice sacrificed after 4 weeks of therapy, the lungs are removed and metastases are enumerated.

### C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. Origin of tumor line: arose spontaneously in 1951 as carcinoma of the lung in a C57BL/6 mouse Cancer Res 15:39, (1955)). See also Malave I et al., J. Natl. Canc. Inst. 62:83-88 (1979).

| | |
|---|---|
| Animals | One sex used for all test and control animals in one experiment. |
| Propagation Strain | C57BL/6 mice |
| Tumor Transfer | Inject cells im in hind leg or implace fragement sc in axillary region with |
| | puncture of inguinal region. Transfer on day 12-14 |
| Testing Strain | BDF1 (C57BL/6 × DBA/2) or C3H mice |
| Time of Transfer | Same as above |
| Weight | Within a 3-g range, minimum weight of 18 g for males and 17 g for females. |
| Exp Size (n) | 6/group for sc implant, or 10/group for im implant.; No. of control groups |
| | varies according to number of test groups. |

### Testing Schedule

| DAY | PROCEDURE |
|---|---|
| 0 | Implant tumor. Prepare materials. Run positive control in every odd-numbered |
| | experiment. Record survivors daily. |
| 1 | Weigh and randomize animals. Begin treatment with therapeutic composition. Typically, |
| | mice receive 1 μg of the test composition in 0.5 ml saline. Controls receive saline alone. |
| | Treatment is one dose/week. Any surviving mice are sacrificed after 4 wks of therapy. |
| 5 | Weigh animals and record. |
| Final day | Kill all survivors and evaluate experiment. |

Quality Control: Acceptable im tumor weight on Day 12 is 500-2500 mg. Acceptable im tumor MedST is 18-28 days. Positive control compound is cyclophosphamide: 20 mg/kg/injection, qd, Days 1-11. Check control deaths, no takes, etc.

Evaluation: Compute mean animal weight when appropriate, and at the completion of testing compute T/C for all test groups. When the parameter is tumor weight, a reproducible T/C of 42% is considered necessary to demonstrate activity. When the parameter is survival time, a reproducible T/C of 125% is considered necessary to demonstrate activity. For confirmed activity a composition must have two multi-dose assays

### D. 3LL Lewis Lung Carcinoma Metastasis Model

This model has been utilized by a number of investigators. See, for example, Gorelik, E. et al., J. Natl. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J E et al., J. Nat'l. Canc. Inst. 69:975-980 (1982); Hilgard, P. et al., Br. J. Cancer 35:78-86 (1977)).

Mice: male C57BL/6 mice, 2-3 months old. Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days.

In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

### Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ^{125I}dUrd into lung cells (Thakur M L et al., J. Lab. Clin. Med. 89:217-228 (1977)). Ten days following tumor amputation, 25 mg of ¹²⁵IdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴-10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

### E. Walker Carcinosarcoma 256

Summary: Tumor may be implanted sc in the axillary region as a 2-6 mm fragment im in the thigh as a 0.2-ml inoculum of tumor homogenate containing 10⁶ viable cells, or ip as a 0.1-ml suspension containing 10⁶ viable cells. Origin of tumor line: arose spontaneously in 1928 in the region of the mammary gland of a pregnant albino rat (J Natl Cancer Inst 13:1356, (1953)).

| | |
|---|---|
| Animals | One sex used for all test and control animals in one experiment. |
| Propagation Strain | Random-bred albino Sprague-Dawley rats |
| Tumor Transfer | S.C. fragment implant is by trochar or 12-g needle into axillary region with |
| | puncture in inguinal area. I.m. implant is with 0.2 ml of tumor homogenate |
| | (containing 10⁶ viable cells) into the thigh. I.p. implant is with 0.1 ml |
| | suspension (containing 10⁶ viable cells) |
| | Day 7 for im or ip implant; Days 11-13 for sc implant |
| Testing Strain | Fischer 344 rats or random-bred albino rats |
| Time of Transfer | Same as above |
| Weight | 50-70 g (maximum of 10-g weight range within each experiment) |
| Exp Size (n) | 6/roup; No. of control groups varies according to number of test groups. |

| | Prepare drug | Administer | Weigh animals | Evaluate on |
|---|---|---|---|---|
| Test system | on day: | drug on days: | on days | days |
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

In addition the following general schedule is followed

| DAY | PROCEDURE |
|---|---|
| 0 | Implant tumor. Prepare materials. Run positive control in every odd-numbered experiment. |
| | Record survivors daily. |
| 1 | Weigh and randomize animals. Begin treatment with therapeutic composition. Typically, |
| | mice receive 1 μg of the test composition in 0.5 ml saline. Controls receive saline alone. |
| | Treatment is one dose/week. Any surviving mice are sacrificed after 4 wks of therapy. |
| Final day | Kill all survivors and evaluate experiment. |

Quality Control: Acceptable i.m. tumor weight or survival time for the above three test systems are: 5WA16: 3-12 g.; 5WA12: 3-12 g.; 5WA31 or 5WA21: 5-9 days.

Evaluation: Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity

### F. A20 Lymphoma

10⁶ murine A20 lymphoma cells in 0.3 ml saline are injected subcutaneously in Balb/c mice. Tumor growth is monitored daily by physical measurement of tumor size and calculation of total tumor volume. After 4 weeks of therapy the mice are sacrificed. Having now generally described the invention, the same will be more readily understood through reference to the following examples which are provided by way of illustration, and are not intended to be limiting of the present invention, unless specified.

### Results of Treatments for Therapeutic Agents

Efficacy of therapeutic heme or heme conjugates or polypeptides such as wild type, mutant, variant superantigens, modified superantigens such as SEG described herein, superantigen fragments such as SEB₁₉₁₋₂₂₀, and superantigens fused to a fusion partner, e.g., tumor targeting polypeptide such as antibody, antibody fragment, receptor or receptor ligand) or therapeutic SS RBCs, liposomes or SS RBC ghosts as described herein in the tumor models described above is shown below. The results of these treatments in tumor models disclosed above shown in the Table below are for each therapeutic composition and dose tested. The results are statistically significant compared to untreated controls using the Wilcoxon rank sum test.

TABLE VI

| Tumor Model | Parameter | % of Control Response |
|---|---|---|
| L1210 | Mean survival time | >130% |
| P388 | Mean survival time | >130% |
| B16 | Mean survival time | >130% |
| B16 metastasis | Median number of metastases | <70% |
| 3LL | Mean survival time | >130% |
| | Mean tumor weight | <40% |
| 3LL metastasis | Median survival time | >130% |
| | Mean lung weight | <60 |
| | Median number of metastases | <60% |
| | Median volume of metastases | <60% |
| | Medial volume of metastases | <60% |
| | Median uptake of IdUrd | <60% |
| Walker carcinoma | Median survival time | >130% |
| | Mean tumor weight | <40% |
| A20 | Mean survival time | >130% |
| | Mean tumor volume | <40% |

### Example 1

### Clinical Trial of Superantigen Compositions

All patients treated have histologically confirmed malignant disease including carcinomas, sarcomas, melanomas, gliomas, neuroblastomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

The SEs means collectively wild type SEs, fragments, homologues or fusion proteins described herein. An SE refers to any one of these individually. An SE is administered intravenously infusion, injection, instillation or implantation before, together with or after heme in doses of 0.01 pg-100 ng. It may be administered every 2-7 days for up to 10 doses. It may be continued daily for up to 3 days after each infusion. The treatments are generally given every 2-7 days for a total of 1-12 treatments. However, the treatment schedule is flexible and may be given for a longer of shorter duration depending upon the patients' response.

Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al., supra)

The efficacy of the therapy in a patient population is evaluated using conventional statistical methods, including, for example, the Chi Square test or Fisher's exact test. Long-term changes in and short term changes in measurements are evaluated separately.

| RESPONSE | DEFINITION |
|---|---|
| Complete remission (CR) | Disappearance of all evidence of disease |
| Partial remission (PR) | □50% decrease in the product of the two greatest perpendicular tumor |
| | diameters; no new lesions |
| Less than partial | 25%-50% decrease in tumor size, stable for at least 1 month |
| remission (<PR) | |
| Stable disease | <25% reduction in tumor size; no progression or new lesions |
| Progression | ≧25% increase in size of any one measured lesion or appearance of |
| | new lesions despite stabilization or remission of disease in other |
| | measured sites |

### Results

A total of 1231 patients are patients treated. The number of patients for each tumor type and the results of treatment are summarized in Table 10. Positive tumor responses are observed in as high as 75-90%% of the patients with breast, gastrointestinal, lung, prostate, renal and bladder tumors as well as melanoma and neuroblastoma.

One thousand and thirteen patients with all tumors exhibit objective clinical responses for an overall response rate of 82%. Tumors generally start to diminish and objective remissions are evident after four weeks of combined SAgs and chemotherapy. Responses endure for an average of 24 months.

TABLE 10

| | | | % of Patients |
|---|---|---|---|
| Patients/Tumors | No. | Response | Responding |
| All patients | 898 | CR | 72.0 |
| | 98 | PR | 8.0 |
| | 24 | <PR | 3.4 |
| | | | % of Patients |
| Tumor Type | No. | Response | Responding |
| Breast adenocarcinoma | 100 | CR + PR + <PR | 80% |
| Gastrointestinal carcinoma | 100 | CR + PR + <PR | 85% |
| Lung Carcinoma | 150 | CR + PR + <PR | 90% |
| Brain glioma/astrocytoma | 50 | CR + PR + <PR | 80% |
| Prostate Carcinoma | 100 | CR + PR + <PR | 80% |
| Lymphoma/Leukemia | 80 | CR + PR + <PR | 75% |
| Head and Neck Cancer | 80 | CR + PR + <PR | 75% |
| Renal and Bladder Cancer | 50 | CR + PR + <PR | 90% |
| Melanoma | 50 | CR + PR + <PR | 80% |
| Neuroblastoma | 50 | CR + PR + <PR | 80% |
| Prostate carcinoma | 100 | CR + PR + <PR | 82% |
| Uterine/Cervical | 100 | CR + PR + <PR | 75% |

Toxicity consists of mild short-lived fever, fatigue and anorexia not requiring treatment. The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

| | | | % of Patients |
|---|---|---|---|
| Patients/Tumors | No. | Response | Responding |
| All patients | 1048 | CR + PR | 72.0 |
| | | | % of Patients |
| Tumor Type | No. | Response | Responding |
| Breast adenocarcinoma | 100 | CR + PR + <PR | 88% |
| Gastrointestinal carcinoma | 100 | CR + PR + <PR | 87% |
| Lung Carcinoma | 150 | CR + PR + <PR | 92% |
| Brain glioma/astrocytoma | 50 | CR + PR + <PR | 87% |
| Prostate Carcinoma | 100 | CR + PR + <PR | 81% |
| Lymphoma/Leukemia | 100 | CR + PR + <PR | 79% |
| Head and Neck Cancer | 100 | CR + PR + <PR | 75% |
| Renal and Bladder Cancer | 50 | CR + PR + <PR | 90% |
| Melanoma | 50 | CR + PR + <PR | 87% |
| Neuroblastoma | 50 | CR + PR + <PR | 83% |
| Prostate carcinoma | 100 | CR + PR + <PR | 86% |
| Uterine/Cervical | 100 | CR + PR + <PR | 85% |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed.

### Example 2

The SE-OX-40L (or 4-1BB)-tumor specific Fv conjugate or SE-mAb Fab-tumor-specific

Fv conjugates described above are administered parenterally, intratumorally, intrathecally, intraperitoneally, intrapleurally by infusion or injection in conventional or sustained release vehicles as given in Section 66 of U.S. patent application Ser. No. 10/428,817, filed May 5, 2003 (incorporated in entirety by reference) in dosages of 0.01 ng/kg to 100 μg/kg using protocols given in Examples 5, 7, 14, 15, 16, 18-23, 38 of U.S. patent application Ser. No. 10/428,817, filed May 5, 2003 (incorporated in entirety by reference).

U.S. Ser. No. 10/428, 817 (Incorporated by Reference)

### Example 5, Page 118

### Construction of Expression Plasmids and Detection of Fusion Proteins

1. The appropriate pUR (or pEX or pMR100) vector is ligated in-frame to cDNA fragments to be expressed as fusion partners using the above plasmids to create an in-frame fusion. cDNA encoding the verotoxins may be obtained from Dr. G. Lingwood, University of Toronto; murine p31 Ii are from Dr. R. Germain, National Institutes of Health and J. Miller, University of Chicago.
2. Bacteria of the following strains are transformed: E. coli K12 71/18 or JM1O3 with pUR vectors, M5219 with pEX vectors or LG9O for pMR100 vectors. The cells are plated on LB medium containing ampicillin (100 mg/ml) and incubated overnight at 37° C. (or 30° C. in the case of the pEX vector). MacConkey lactose indicator plates should be used for pMR100.
3. Individual colonies are tested for the presence of the desired insert by plasmid minipreps.

If most of the colonies can be assumed to contain a cDNA (because directional cloning or a dephosphorylated vector was used in step 1), they can be screened for protein production in parallel (see step 4b). If not, clones that contain a cDNA, as determined by plasmid minipreps, can be screened for protein expression later. cDNA inserts into a pMR100 plasmid can be detected readily as red colonies on the MacConkey lactose indicator plates.

4. Colonies are screened as follows for expression of the fusion protein.
a. Grow small cultures from 5-10 colonies in LB medium containing ampicillin (100 mg/ml). Incubate overnight at 37° C. (or at 30° C. for pEX).
b. Inoculate 5 ml of LB medium containing ampicillin (100 mg/ml) with 50 ml of each overnight culture: Incubate for 2 hours at 37° C. (or at 30° C. for pEX) with aeration. Remove 1 ml of uninduced culture, place it in a microfuge tube, and process as described in steps d and e. If screening for protein production is being done in parallel, prepare plasmid minipreps from 1-ml aliquots of the overnight cultures.
c. Induce each culture as follows: For pUR or pMR100 vectors, add isopropylthio-b-D-galactoside (IPTG) to a final concentration of 1 nM and continue incubation at 37° C. with aeration. For pEX vectors, transfer the culture to 40° C. and continue incubating with aeration.
d. At various time points during the incubation (i.e., 1, 2, 3, and 4 hours), transfer 1 ml of each culture to a microfuge tube, and centrifuge at 12,000 g for 1 minute at room temperature in a microfuge. Remove the supernatant by aspiration. The kinetics of induction varies with different proteins, so it is necessary to determine the time at which the maximum amount of product is produced.
e. Resuspend each pellet in 100 ml of 1×SDS gel-loading buffer, heat to 100° C. for 3 minutes, and then centrifuge at 12,000 g for 1 minute at room temperature. Load 15 ml of each suspension on a 6% SDS polyacrylamide gel. Use suspensions of cells containing the vector alone as a control. (For pEX and ORF vectors, also use b-galactosidase as a control.) The fusion protein should appear as a novel band migrating more slowly than the intense b-galactosidase band in the control. It is not uncommon for a protein the size of b-galactosidase to be present along with the fusion protein.

### Composition of 1×SDS Gel-Loading Buffer

### 50 mM Tris Cl (pH 6.8)

100 mM dithiothreitol (DTT)
2% SDS (electrophoresis grade)
0.1% bromophenol blue
10% glycerol
1×SDS gel-loading buffer lacking dithiothreitol can be stored at room temperature.
Dithiothreitol should then be added, just before the buffer is used, from a 1 M stock.
U.S. Ser. No. 10/428,817 (Incorporated by Reference)

### Example 20, Page 149-158

### General Test Evaluation Procedures for Constructs and SAg Activated Effector T or NKT Cells

### I. General Test Evaluation Procedures

### A. Calculation of Mean Survival Time

Mean survival time is calculated according to the following formula:

$$Mean survival time ( days ) = \frac{S + AS ( A - 1 ) - ( B + 1 ) NT}{S ( A - 1 ) - NT}$$

### DEFINITIONS

Day: Day on which deaths are no longer considered due to drug toxicity. Example: with treatment starting on Day 1 for survival systems (such as L1210, P388, B16, 3LL, and W256):

### Day A: Day 6.

Day B: Day beyond which control group survivors are considered “no-takes.”
Example: with treatment starting on Day 1 for survival systems (such as L1210, P388, and W256), Day B-Day 18. For B16, transplanted AKR, and 3LL survival systems, Day B is to be established.
S: If there are “no-takes” in the treated group, S is the sum from Day A through Day B. If there are no “no-takes” in the treated group, S is the sum of daily survivors from Day A onward.
S(A−1):Number of survivors at the end of Day (A−1).

Example: for 3LE21, S(A−1)=number of survivors on Day 5.

NT: Number of “no-takes” according to the criteria given in Protocols 7.300 and 11.103.

### B. T/C Computed for all Treated Groups

T/C is the ratio (expressed as a percent) of the mean survival time of the treated group divided by the mean survival time of the control group. Treated group animals surviving beyond Day B, according to the chart below, are eliminated from calculations:

| No. of survivors in | Percent of “no-takes” | |
|---|---|---|
| treated group beyond Day B | in control group | Conclusion |
| 1 | Any percent | “no-take” |
| 2 | <10 | drug inhibition |
| | 10 | “no-takes” |
| 3 | <15 | drug inhibition |
| | 15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

### Calculation of Median Survival Time

Median Survival Time is defined as the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there is no deletion of early deaths or survivors, with the following exception:

C. Computation of Median Survival Time from Survivors

If the total number of animals including survivors (N) is even, the median survival time (days) (X+Y)/2, where X is the earlier day when the number of survivors is N/2, and Y is the earliest day when the number of survivors (N/2)−1. If N is odd, the median survival time (days) is X.

D. Computation of Median Survival Time from Mortality Distribution If the total number of animals including survivors (N) is even, the median survival time (days) (X+Y)/2, where X is the earliest day when the cumulative number of deaths is N/2, and Y is the earliest day when the cumulative number of deaths is (N/2)+1. If N is odd, the median survival time (days) is X.

Cures and “No-Takes”: “Cures” and “no-takes” in systems evaluated by median survival time are based upon the day of evaluation. On the day of evaluation any survivor not considered a “no-take” is recorded as a “cure.” Survivors on day of evaluation are recorded as “cures” or “no-takes,” but not eliminated from the calculation of the median survival time.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times {( width [ mm ] )}^{2}}{2} Or \frac{L \times {( W )}^{2}}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of small tumors | Percent of “no-takes” | |
|---|---|---|
| in treated group | in control group | Action |
| 17 | Any percent | no-take; not used in |
| | | calculations |
| 18-39 | <10 | drug inhibition; use in |
| | | calculations |
| | 10 | no-takes; not used in |
| | | calculations |
| 40 | <15 | drug inhibition; use in |
| | | calculations |
| | 15 | Code all nontoxic tests |
| | | “33” |

Positive control compounds are not considered to have “no-takes” regardless of the number of “no-takes” in the control group. Thus, the tumor weights of all surviving animals are used in the calculation of TIC for the positive control. T/C is computed for all treated groups having more than 65% survivors. The T/C is the ratio (expressed as a percent) of the mean tumor weight for treated animals divided by the mean tumor weight for control animals. SDs of the mean control tumor weight are computed the factors in a table designed to estimate SD using the estimating factor for SD given the range (difference between highest and lowest observation). Biometrik Tables for Statisticians (Pearson E S, and Hartley H G, eds.) Cambridge Press, vol. 1, table 22, p. 165.

### II. Specific Tumor Models

### A. Lymphoid Leukemia L1210

Summary: Ascitic fluid from donor mouse is transferred into recipient BDF1 or CDF1 mice. Treatment begins 24 hours after implant. Results are expressed as a percentage of control survival time. Under normal conditions, the inoculum site for primary screening is i.p., the composition being tested is administered i.p., and the parameter is mean survival time. Origin of tumor line: induced in 1948 in spleen and lymph nodes of mice by painting skin with MCA. J Natl Cancer Inst. 13:1328, 1953.

### Animals

Propagation: DBA/2 mice (or BDF1 or CDF1 for one generation).
Testing: BDF1 (C57BL/6×DBA/2) or CDF1 (BALB/c×DBA/2) mice.
Weight: Within a 3-g weight range, with a minimum weight of 18 g for males and 17 g for females.
Sex: One sex used for all test and control animals in one experiment.
Experiment Size Six animals per test group.
Control Groups Number of animals varies according to number of test groups.

### Tumor Transfer

Inject i.p., 0.1 ml of diluted ascitic fluid containing 10⁵ cells.

### Time of Transfer for Propagation: Day 6 or 7.

### Time of Transfer for Testing: Day 6 or 7.

### Testing Schedule

Day 0: Implant tumor. Prepare materials. Run positive control in every odd-numbered experiment. Record survivors daily.
Day 1: Weigh and randomize animals. Begin treatment with therapeutic composition.

Typically, mice receive 1 μg of the test composition in 0.5 ml saline. Controls receive saline alone. The treatment is given as one dose per week. Any surviving mice are sacrificed after 4 weeks of therapy.

Day 5: Weigh animals and record.
Day 20: If there are no survivors except those treated with positive control compound, evaluate study.
Day 30: Kill all survivors and evaluate experiment.

### Quality Control

Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%

### Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

### B. Lymphocytic Leukemia P388

Summary: Ascitic fluid from donor mouse is implanted in recipient BDF1 or CDF1 mice. Treatment begins 24 hours after implant. Results are expressed as a percentage of control survival time. Under normal conditions, the inoculum site for primary screening is ip, the composition being tested is administered ip daily for 9 days, and the parameter is median survival time. Origin of tumor line: induced in 1955 in a DBA/2 mouse by painting with MCA. Scientific Proceedings, Pathologists and Bacteriologists 33:603, 1957.

### Animals

Propagation: DBA/2 mice (or BDF1 or CDF1 for one generation)
Testing: BDF1 (C57BL/6×DBA/2) or CDF1 (BALB/c×DBA/2) mice.
Weight: Within a 3-g weight range, with a minimum weight of 18 g for males and 17 g for females.
Sex: One sex used for all test and control animals in one experiment.
Experiment Size Six animals per test group.
Control Groups Number of animals varies according to number of test groups.

### Tumor Transfer

### Implant: Inject ip

Size of Implant: 0.1 ml diluted ascitic fluid containing 10⁶ cells.

### Time of Transfer for Propagation: Day 7.

### Time of Transfer for Testing: Day 6 or 7.

### Testing Schedule

Day 0: Implant tumor. Prepare materials. Run positive control in every odd-numbered experiment. Record survivors daily.
Day 1: Weigh and randomize animals. Begin treatment with therapeutic composition. Typically, mice receive 1 ug of the composition being tested in 0.5 ml saline. Controls receive saline alone. The treatment is given as one dose per week. Any surviving mice are sacrificed after 4 weeks of therapy.
Day 5: Weigh animals and record.
Day 20: If there are no survivors except those treated with positive control compound, evaluate experiment.
Day 30: Kill all survivors and evaluate experiment.

### Quality Control

Acceptable median survival time is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

### Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a synthetic must have two multi-dose assays (each performed at a different laboratory) that produce a T/C 125%; a natural product must have two different samples that produce a T/C 125% in multi-dose assays.

### C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The composition being tested is administered ip, and the parameter is mean survival time. Origin of tumor line: arose spontaneously in 1954 on the skin at the base of the ear in a C57BL/6 mouse. Handbook on Genetically Standardized Jax Mice. Roscoe B. Jackson Memorial Laboratory, Bar Harbor, Me., 1962. See also Ann NY Acad Sci 100, Parts 1 and 2, 1963.

### Animals

Propagation: C57BL/6 mice.
Testing: BDF1 (C57BL/6×DBA/2) mice.
Weight: Within a 3-g weight range, with a minimum weight of 18 g for males and 17 g for females.
Sex: One sex used for all test and control animals in one experiment.

Experiment Size Ten animals per test group. For control groups, the number of animals varies according to number of test groups.

### Tumor Transfer

Propagation: Implant fragment sc by trochar or 12-gauge needle or tumor homogenate (see below) every 10-14 days into axillary region with puncture in inguinal region.
Testing: Excise sc tumor on Day 10-14.
Homogenate: Mix 1 g or tumor with 10 ml of cold balanced salt solution and homogenize, and implant 0.5 ml of this tumor homogenate ip or sc.
Fragment: A 25-mg fragment may be implanted sc.

### Testing Schedule

Day 0: Implant tumor. Prepare materials. Run positive control in every odd-numbered experiment. Record survivors daily.
Day 1: Weigh and randomize animals. Begin treatment with therapeutic composition.

Typically, mice receive 1 mg of the composition being tested in 0.5 ml saline. Controls receive saline alone. The treatment is given as one dose per week. Any surviving mice are sacrificed 8 weeks of therapy.

Day 5: Weigh animals and record.
Day 60: Kill all survivors and evaluate experiment.

### Quality Control

Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

### Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a therapeutic composition should have two multi-dose assays that produce a T/C 125%.

Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. The treatment is given as one dose per week. Mice sacrificed after 4 weeks of therapy, the lungs are removed and metastases are enumerated.

### C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. The composition being tested is administered ip daily for 11 days and the results are expressed as a percentage of the control. Origin of tumor line: arose spontaneously in 1951 as carcinoma of the lung in a C57BL/6 mouse. Cancer Res 15:39, 1955. See, also Malave, I. et al., J. Nat'l. Canc. Inst. 62:83-88 (1979).

### Animals

Propagation: C57BL/6 mice.
Testing: BDF1 mice or C3H.
Weight: Within a 3-g weight range, with a minimum weight of 18 g for males and 17 g for females.
Sex: One sex used for all test and control animals in one experiment.

Experiment Size Six animals per test group for sc implant, or ten for im implant. For control groups, the number of animals varies according to number of test groups.

### Tumor Transfer

Implant: Inject cells im in hind leg or implant fragment sc in axillary region with puncture in inguinal region.

### Time of Transfer for Propagation: Days 12-14.

### Time of Transfer for Testing: Days 12-14.

### Testing Schedule

Day 0: Implant tumor. Prepare materials. Run positive control in every odd-numbered experiment. Record survivors daily.
Day 1: Weigh and randomize animals. Begin treatment with therapeutic composition. Typically, mice receive 1 ug of the composition being tested in 0.5 ml saline. Controls receive saline alone. The treatment is given as one dose per week. Any surviving mice are sacrificed after 4 weeks of therapy.
Day 5: Weigh animals and record.
Final Day: Kill all survivors and evaluate experiment.

### Quality Control

Acceptable im tumor weight on Day 12 is 500-2500 mg. Acceptable im tumor median survival time is 18-28 days. Positive control compound is cyclophosphamide: 20 mg/kg/injection, qd, Days 1-11. Check control deaths, no takes, etc.

### Evaluation

Compute mean animal weight when appropriate, and at the completion of testing compute

T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity a synthetic must have two multi-dose assays (each performed at a different laboratory); a natural product must have two different samples.

### D. 3LL Lewis Lung Carcinoma Metastasis Model

This model has been utilized by a number of investigators. See, for example, Gorelik, E. et al., J. Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J. E. et al., J. Nat'l. Canc. Inst. 69:975-980 (1982); Hilgard, P. et al., Br. J. Cancer 35:78-86 (1977)).

Mice: male C57BL/6 mice, 2-3 months old.

Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days.

Typically, mice receive 1 ug of the composition being tested in 0.5 ml saline. Controls receive saline alone. The treatment is given as one or two doses per week. In experiments involving tumor excision, mice with tumors 8-10 mm in diameter are divided into two groups. In one group, legs with tumors are amputated after ligation above the knee joints. Mice in the second group are left intact as nonamputated tumor-bearing controls.

Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M. L. et al., J. Lab. Clin. Med. 89:217-228 (1977). Ten days following tumor amputation, 25 mg of FdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 1×10⁶ 3LL cells. Amputation of tumors produced following inoculation of 1×10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴-1×10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

### E. Walker Carcinosarcoma 256

Summary: Tumor may be implanted sc in the axillary region as a 2-6 mm fragment, im in the thigh as a 0.2-ml inoculum of tumor homogenate containing 10⁶ viable cells, or ip as a 0.1-ml suspension containing 10⁶ viable cells. Treatment of the composition being tested is usually ip. Origin of tumor line: arose spontaneously in 1928 in the region of the mammary gland of a pregnant albino rat. J Natl Cancer Inst 13:1356, 1953.

### Animals

Propagation: Random-bred albino Sprague-Dawley rats.
Testing: Fischer 344 rats or random-bred albino rats.
Weight Range: 50-70 g (maximum of 10-g weight range within each experiment).
Sex: One sex used for all test and control animals in one experiment.

Experiment Size Six animals per test group. For control groups, the number of animals varies according to number of test groups.

### Time of Tumor Transfer

Time of Transfer for Propagation: Day 7 for im or ip implant; Days 11-13 for sc implant.
Time of Transfer for Testing: Day 7 for im or ip implant; Days 11-13 for sc implant.

### Tumor Transfer

Sc fragment implant is by trochar or 12-gauge needle into axillary region with puncture in inguinal area. Im implant is with 0.2 ml of tumor homogenate (containing 10⁶ viable cells) into the thigh. Ip implant is with 0.1 ml of suspension (containing 10⁶ viable cells) into the ip cavity.

### Testing Schedule

Prepare and administer compositions under test on days, weigh animals, and evaluate test on the days listed in the following tables.

| Test system | Prepare drug | Administer drug | Weigh animals | Evaluate |
|---|---|---|---|---|
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

Day 0: Implant tumor. Prepare materials. Run positive control in every odd-numbered experiment. Record survivors daily.

Day 1: Weigh and randomize animals.

Final Day: Kill all survivors and evaluate experiment.

### Quality Control

Acceptable im tumor weight or survival time for the above three test systems: 5WA16: 3-12 g. 5WA12: 3-12 g. 5WA31 or 5WA21: 5-9 days.

### Evaluation

Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity a therapeutic agent must have activity in two multi-dose assays.

### F. A20 Lymphoma

10⁶ murine A20 lymphoma cells in 0.3 ml saline are injected subcutaneously in Balb/c mice. The mice are treated intravenously with 1 g of the composition being tested in 0.5 ml saline. Controls receive saline alone. The treatment is given as one dose per week. Tumor growth is monitored daily by physical measurement of tumor size and calculation of total tumor volume. After 4 weeks of therapy the mice are sacrificed.

### Treatment Regimens and Results (Constructs)

For determining efficacy in the tumor models described above the general categories of therapeutic constructs used are given below. For all of the classes of conjugates listed below, the SAg component can be prepared as either a DNA encoding SAg or as the SAg polypeptide itself. In either form the SAg DNA or protein may be conjugated to additional molecules, either nucleic acid or polypeptides. Operationally, for therapeutic use in vivo or ex vivo, these conjugates may be prepared by chemical coupling or by recombinant means (whichever is appropriate) and conjugated to a tumor-targeting structure or incorporated into a vehicle (e.g., liposomes) that themselves comprise a tumor targeting structure(s). Again, examples of such targeting structures include, but are not limited to, an antibody, antigen, receptor or receptor ligand. Methods are disclosed in Examples 1, 3, 4, 5, 6, 7, 14, 17, 18, 30-32.
1. SAg Nucleic Acid Constructs including Phage Displays and SAg Transfected Bacterial Cells
2. Glycosylated SAgs
3. Chimeric SAgs

Conjugates having a Superantigen component (polypeptide or nucleic acid) and a partner that is either a single component or a conjugate of 2 or more components (protein, carbohydrate, lipid or DNA) as indicated below.

| Superantigen | Partner |
|---|---|
| (Protein or DNA) | (Single Component or Conjugate) |
| 4. | DNA coding sequence |
| 5. | Polypeptide |
| 6. | Nucleic acid |
| 7. | Tumor associated Peptide |
| 8. | Tumor Antigen-MHC protein |
| 9. | LPS |
| 10. | Lipoarabinomannan |
| 11. | Ganglioside |
| 12. | Glycosphingolipid |
| 13. | Ganglioside-CD1 receptor |
| 14. | Glycosphingolipid-CD1 receptor |
| 15. | Glycosylceramide (e.g., Gal-Cer) |
| 16. | GalCer-CD1 receptor |
| 17. | Gal |
| 18. | Arg-Gly-Asp or Asn-Gly-Arg |
| 19 | iNOS |
| 20. | Gb2 or Gb3 or Gb4 |
| 21. | (Gb2 or Gb3 or Gb4)-CD1 receptor. |
| 22. | -GPI-(Gb2 or Gb3 or G4) |
| 23. | -GPI-(Gb2 or Gb3 or Gb4)-CD1 receptor |
| 24. | Verotoxin |
| 25. | Verotoxin A or B Subunit |
| 26. | IFNa receptor peptide homologous to VT |
| 27. | CD19 peptide homologous to VT |
| 28. | LDL, VLDL, HDL, IDL |
| 29. | Apolipoproteins (e.g., Lp(a), apoB-100, apoB-48, apoE) |
| 30. | OxyLDL, oxyLDL mimics, (e.g., 7b-hydroperoxycholesterol, |
| | 7b-hydroxycholesterol, 7-ketocholesterol, 5a-6a- |
| | epoxycholesterol, 7b-hydroperoxy-choles-5-en-3b-ol, 4- |
| | hydroxynonenal(4-HNE), 9-HODE, 13-HODE and cholesterol-9- |
| | HODE) |
| | OxyLDL by products (e.g. lysolecithin, |
| 31. | lysophosphatidylcholine, malondialdehyde, 4- |
| | hydroxynonenal) |
| 32. | LDL & oxyLDL receptors (e.g., LDL oxyLDL, acetyl-LDL, |
| | VLDL, LRP, CD36, SREC, LOX-1, macrophage |
| scavenger | receptors) |
| 33. | phytosphingosine, -GPI-phytosphingosine |
| 34. | tumor associated lipid antigens |
| 35. | glycolipid, proteolipid, glycosphingolipid, sphingolipid |
| | with inositolphosphate -containing head groups, |
| | phytoglycolipids, mycoglycolipids, -GPI- |
| sphingosines, | -GPI-lipids |
| 36. | sphingolipids with inositolphosphate-containing head |
| | groups having the general structure: |
| | ceramide-P-myoinositol-X with X referring to |
| polar | substituents comprising ceramide-p- |
| inositol-mannose, | inositol-1-P-(6)mannose(a1, 2 |
| inosito1-1P-(1)ceramide, | (inositol-P)2-ceramide, |
| inositol-P-inositol-P-ceramide, | inositol-P-inositol-P- |
| ceramide. | |
| 37. | tumor associated glycan antigens consisting of |
| | peptidoglycans or glycan phosphotidyinositol (GPI) |
| | structures |

### Use in Established Tumors

For proteins or nucleic acid constructs, treatment consists of injecting animals, iv or ip with 50, 500 1000 or 5,000 ng of in 0.1-0.5 ml of normal saline. Unless indicated otherwise above, treatments are given one to three times per week for two to five weeks. Phage displays, yeast displays and vesilcle, SAg-bacterial or viral constructs or SAg vesicles are administered as 10⁹ transducing units (TU) and irradiated bacterial cells, yeast cells as 10⁵-10⁶ cells iv into the tail vein one to three times per week for two to five weeks or directly into tumor in 30-75% or the iv doses on the same schedule. Exosomes or vesicles, harvested from transfected, transformed or fusion tumor cells or sickled cells or mutant yeast are given i.v. into the tail vein in a dose of 0.25-1 g per animal one to three times per week for two to five weeks. The results shown in Table VI are for each composition and dose tested. The results are statistically significant by the Wilcoxon rank sum test.

Treatment regimens for SAg activated effector T or NKT cells are in Example 16, 18, 19. The preferred animal model for evaluation of the adoptively transferred T or NKT effector cells is the MCA 205/207 fibrosarcoma with pulmonary metastases (Shu S. et al., J. Immunol. 152: 1277-1288 (1994)). The other models given in Example 20 are also suitable for evaluation of the therapeutic effectiveness of the effector T cells.

TABLE VI

| Tumor Model | Parameter | % of Control Response |
|---|---|---|
| L1210 | Mean survival time | >130% |
| P388 | Mean survival time | >130% |
| B16 | Mean survival time | >130% |
| B16 metastasis | Median number of metastases | <70% |
| 3LL | Mean survival time | >130% |
| | Mean tumor weight | <40% |
| 3LL metastasis | Median survival time | >130% |
| | Mean lung weight | <60 |
| | Median number of metastases | <60% |
| | Median volume of metastases | <60% |
| | Medial volume of metastases | <60% |
| | Median uptake of IdUrd | <60% |
| Walker carcinoma | Median survival time | >130% |
| | Mean tumor weight | <40% |
| A20 | Mean survival time | >130% |
| | Mean tumor volume | <40% |

U.S. Ser. No. 10/428,817 (Incorporated by Reference)

### Example 23, Page 158-160

### Example 23

### Treatment Procedures

### Constructs (or Preparations)

Doses of the constructs are determined as described above using, inter alia, appropriate animal models of tumors. Two classes of therapeutic compositions are administered namely SAg proteins or SAg conjugates (nucleic acids or peptides-polypeptides), SAg phage displays, SAg yeast displays, SAg bacterial cell displays, as described above for animal models. A treatment consists of injecting the patient with 0.5-500 mg of Construct intravenously in 200 ml

TABLE VII

| RESPONSE | DEFINITION |
|---|---|
| Complete remission | Disappearance of all evidence of disease |
| (CR) | |
| Partial remission (PR) | >50% decrease in the product of the two greatest |
| | perpendicular tumor diameters; no new lesions |
| Less than partial | 25-50% decrease in tumor size, stable for at least |
| remission (<PR) | 1 month |
| Stable disease | <25% reduction in tumor size; no progression or |
| | new lesions |
| Progression | >25% increase in size of any one measured lesion |
| | or appearance of new lesions despite stabilization |
| or | remission of disease in other measured |
| sites | |

The efficacy of the therapy in a population is evaluated using conventional statistical methods including, for example, the Chi Square test or Fisher's exact test. Long-term changes in and short term changes in measurements can be evaluated separately.

### Results

One hundred and fifty patients are treated. The results are summarized in Table VIII. Positive tumor responses are observed in 80% of the patients as follows:

TABLE VIII

| | All Patients | |
|---|---|---|
| Response | No. | % |
| PR | 20 | 66 |
| <PR | 10 | 33 |
| Tumor Types | Response | % of Patients |
| Breast Adenocarcinoma | PR + <PR | 80 |
| Gastrointestinal Carcinom | PR + <PR | 75 |
| Lung Carcinoma | PR + <PR | 75 |
| Prostate Carcinoma | PR + <PR | 75 |
| Lymphoma/Leukemia | PR + <PR | 75 |
| Head and Neck Cancer | PR + <PR | 75 |
| Renal and Bladder Cance | PR + <PR | 75 |
| Melanoma | PR + <PR | 75 |

All the references, patents and patent applications cited above in this patent application and their references are incorporated by reference in entirety, whether specifically incorporated or not. In addition, the following patent applications and their references are incorporated by reference in their entirety:

| Inventor | Serial No. | Filing Date | Title |
|---|---|---|---|
| Terman, D. S. | 61/455,592 | Oct. 20, 2010 | Compositions and Methods for Treatment of Cancer |
| Terman, D. S | 12/276,941 | Allowance | Compositions and Methods for Treatment of Cancer |
| | | Jun. 27, 2010 | |
| Terman D. S. | 12/276,941 | Nov. 24, 2008 | Compositions and Methods for Treatment of Cancer |
| Terman D. S. | 12/145,949 | Jun. 25, 2008 | Compositions and Methods for Treatment of Cancer |
| Terman D. S. | 10/937,758 | Sep. 8, 2004 | Compositions and Methods for Treatment of Cancer |
| Terman, D. S. | 12/586,532 | Sep. 22, 2009 | Sickled Erythrocytes with Anti-tumor Molecules Induce Tumoricidal |
| | | | Effects |
| Terman, D. S. | 61,215,906 | May 11, 2009 | Sickled Erythrocytes, Nucleated Precursors & Erythroleukemia Cells |
| | | | for Targeted Delivery of Tumoricidal Agents |
| Terman, D. S | 61/211,227 | Mar. 28, 2009 | Sickled Erythrocytes, Nucleated Precursors & Erythroleukemia Cells |
| | | | for Targeted Delivery of Tumoricidal Agents |
| Terman, D. S. | 61/206,338 | Jan. 28, 2009 | Sickled Erythrocytes, Nucleated Precursors & Erythroleukemia Cells |
| | | | for Targeted Delivery of Tumoricidal Agents |
| Terman D. S. | 61/205,776 | Jan. 22, 2009 | Sickled Erythrocytes Induced Tumor Vaso-occlusion and |
| | | | Tumoricidal Effects |
| Terman, D. S. | 61/192,949 | Sep. 22, 2008 | Sickled Erythrocytes, Nucleated Precursors & Erythroleukemia Cells |
| | | | for Targeted Delivery of Oncolytic Viruses, Anti-tumor Proteins, |
| | | | Plasmids, Toxins, Hemolysins and Chemotherapy |
| Terman, D. S. | 61/001,585 | Nov. 1, 2007 | Sickled Erythorcytes, Nucleated Precursors and Erythroleukemia |
| | | | cell for Targeted Delivery of Oncolytic Viruses, Anti-tumor Proteins, |
| | | | siRNAs, Plasmids, Toxins, Hemolysins, Prodrugs and |
| | | | Chemotherapy |
| Terman, D, S. | PCT/US07/69869 | May 29, 2007 | Sickled Erythrocytes, Nucleated Precursors & Erythroleukemia Cells |
| Dewhirst M. W. | | | for Targeted Delivery of Oricolytic Viruses, Anti-tumor Proteins, |
| | | | Plasmids, Toxins, Hemolysins and Chemotherapy |
| Terman, D. S. | 60/842,213 | Sep. 5, 2006 | Sickled Erythrocytes & Nucleated Precursors for Targeted Delivery |
| | | | of Oncolytic Toxins, Viruses, hemolysins and chemotherapy |
| Terman, D. S. | 60/819,551 | Jul. 8, 2006 | Sickled Erythrocytes & Nucleated Precursors for Targeted Delivery |
| | | | of Oncolytic Toxins, Viruses, hemolysins and chemotherapy |
| Terman, D. S. | 60/809,553 | May 30, 2006 | Sickled Erythrocytes & Nucleated Precursors for Targeted Delivery |
| | | | of Oncolytic Toxins, Viruses, hemolysins and chemotherapy |
| Temian, D. S. | 60/799,514 | May 10, 2006 | Synergy of Superantigens, Cytokines and Chemotherapy in |
| Bohach, G | | | Treatment of Malignant Disease |
| Terman, D. S, Etiene, | PCT/US05/022638 | Jun. 27, 2005 | Enterotoxin Gene Cluster Superantigens (egc) to Treat Malignant |
| J., Vandenesch, F., | | | Disease |
| Lina, G. Bohach, G. | | | |
| Terman, D. S, Etiene, | 60/583,692 | Jun. 29, 2004 | Enterotoxin Gene Cluster Superantigens (egc) to Treat Malignant |
| J., Vandenesch, F., | | | Disease |
| Lina, G. Bohach, G. | | | |
| Terman, D. S. | 60/665,654 | Mar. 23,2005 | Enterotoxin Gene Cluster Superantigens (egc) to Treat Malignant |
| | | | Disease |
| Terman, D. S. Etiene, | 60/626,159 | Nov. 6, 2004 | Enterotoxin Gene Cluster Superantigens (egc) to Treat Malignant |
| J., Vandenesch, F., | | | Disease |
| Lina, G. Bohach, G. | | | |
| Terman, D. S | 7,776,822 | Issued | Intrathecal and Intrapleural Superantigens to Treat Malignant |
| | | Aug. 17, 2010 | Disease |
| Terman, D. S. | 60/583,692 | Jun. 29, 2004 | Intrathecal and Intrapleural Superantigens to Treat Malignant |
| | | | Disease |
| Terman, D. S. | 60/550,926 | Mar. 5, 2004 | Intrathecal and Intrapleural Superantigens to Treat Malignant |
| | | | Disease |
| Terman, D. S. | 60/539,863 | Jan. 27, 2004 | Intrathecal and Intrapleural Superantigens to Treat Malignant |
| | | | Disease |
| Terman, D. S. | PCT/US03/14381 | May 8, 2003 | Intrathecal and Intrapleural Superantigens to Treat Malignant |
| | | | Disease |
| Terman, D. S. | 10/428,817 | May 5, 2003 | Composition and Methods for Treatment of Neoplastic Diseases |
| Terman, D. S. | 60/438,686 | Jan. 9, 2003 | Intrathecal and Intrapleural Superantigens to Treat Malignant |
| | | | Disease |
| Terman, D. S. | 60/415,310 | Oct. 1, 2002 | Intrathecal and Intratumoral Superantigens to Treat Malignant |
| | | | Disease. |
| Terman, D. S. | 60/406,750 | Aug. 29, 2002 | Intrathecal Superantigens to Treat Malignant Fluid Accumulation |
| Terman, D. S. | 60/415,400 | Oct. 2, 2002 | Composition and Methods for Treatment of Neoplastic Diseases |
| Terman, D. S. | 60/406,697 | Aug. 28, 2002 | Compositions and Methods for Treatment of Neoplastic Diseases |
| Terman, D. S. | 60/389,366 | Jun. 15, 2002 | Compositions and Methods for Treatment of Neoplastic Diseases |
| Terman, D. S. | 60/378,988 | May 8, 2002 | Compositions and Methods for Treatment of Neoplastic Diseases |
| Terman, D. S. | 09/870,759 | May 30, 2001 | Compositions and Methods for Treatment of Neoplastic Diseases |
| Terman, D. S. | 09/751,708 | Dec. 28, 2000 | Compositions and Methods for Treatment of Neoplastic Diseases |
| Terman, D. S. | 09/640,884 | Aug. 30, 2000 | Compositions and Methods for Treatment of Neoplastic Diseases |
| Terman, D. S. | 60/151,470 | Aug. 30, 1999 | Compositions and Methods for Treatment of Neoplastic Diseases |

Having now fully described this invention, it will be appreciated by those skilled in the art that the same can be performed within a wide range of equivalent parameters, concentrations, and conditions without departing from the spirit and scope of the invention and without undue experimentation.

We claim:

1. A method of treating a subject with a carcinoma comprising administering to said subject parenterally by infusion or injection a tumoricidally effective amount of a composition consisting of:
(i) native staphylococcal enterotoxin G which native protein:
(a) has the biological activity of stimulating T cell mitogensis via a T cell receptor vβregion; or
(ii) a biologically active homologue or fragment of a native staphylococcal enterotoxin or streptococcal pyrogenic exotoxin, which homologue or fragment:
(a) has the biological activity of stimulating T cell mitogenesis via a T cell receptor vβregion and
(b) has sequence homology characterized as a z value exceeding 13
when the sequence of the homologue or said fragment is compared to the sequence of a native staphylococcal enterotoxin or a native streptococcal pyrogenic exotoxin, determined by FASTA analysis using gap penalties of −12 and −2, Blosum 50 matrix and Swiss-PROT or PIR database; or
(iii) a biologically active fusion protein having said biological activity and said sequence homology, comprising
(A) said homologue,
(B) a native staphylococcal enterotoxin,
(C) a native streptococcal pyrogenic exotoxin, or
(D) a biologically active fragment of said homologue, said native enterotoxin or said native exotoxin,
fused to a peptide or polypeptide fusion partner.

2. A fusion protein according to claim 1 wherein said fusion partner is selected from a group comprising an antibody or antibody fragment specific for tumor cells, tumor vasculature or tumor stroma expressing erb/neu, MUC1, 5T4, endoglin, TGFβ. receptor, E-selectin, P-selectin, VCAM-1, ICAM-1, PSMA, a VEGF/VPF receptor, a FGF receptor, a TIE, αᵥβ₃ integrin, a pleiotropin, an endosialin, cytokine-inducible or coagulant-inducible products of intratumoral blood vessels, aminophospholipids, phosphatidylserine or phosphatidylethanolamine.

3. A fusion protein according to claim 1 wherein said fusion partner is selected from a group comprising polypeptides consisting of costimulatory molecules OX-40L or 4-1BBL alone or fused to a tumor specific antibody fragment.


SEQUENCE LISTING (23 sequences)

SEQ ID NO: 1 - protein, 233 aa, Staphylococcus aureus
QPDLKLDELNKVSDYKNNKGTMGNVMNLYMSPPVEGRGVINSRQFLSHDLIFPIEYKSYNEVKTELENTELANNYGKKVDIFGVPYFYTCIIPKSEPDINQNFGGCCMYGGLTFNSSENERDKLITVQVTIDNRQSLGFTITTNKNMVTIQELDYKARHWTLEKEKKLYEFDGSAFESGYIKFTEKNNTSFWFDLFPKKELVPFVPYKFLNIYGDNKVVDSKSIKMEVFLNTH

SEQ ID NO: 2 - protein, 233 aa, Staphylococcus aureus
KPDPRPGELNRVSDYKKNKGTMGNVESLYKDKAVIAENVKNTRQFLGHDLIFPIPYSEYKEVKSEFINKKTADKFKDKRLDVFGIPYFYTCLVPKNESREEFIFDGVCIYGGVTMHSTADSISKNIIVPVTVDNKQQFSFTISTNKKTVTVQELDYKVRNWLTNNKKLYEFDGSAYETGYIKFIEQNKDSFWYDLFPKKDLVPFIPYKFVNIYGDNKTIDASSVKIEVHLTTM

SEQ ID NO: 3 - protein, 239 aa, Staphylococcus aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 4 - protein, 234 aa, Staphylococcus aureus
SSQPDPTPEQLNKSSQFTGVMGNLRCLYDNHFVEGTNVRSTGQLLQHDLIFPIKDLKLKNYDSVKTEFNSKDLAAKYKNKDVDIFGSNYYYNCYYSEGNSCKNAKKTCMYGGVTEHHRNQIEGKFPNITVKVYEDNENILSFDITTNKKQVTVQELDCKTRKILVSRKNLYEFNNSPYETGYIKFIESSGDSFWYDMMPAPGAIFDQSKYLMLYNDNKTVSSSAIAIEVHLTKK

SEQ ID NO: 5 - protein, 221 aa, Staphylococcus aureus
QQDPDPSQLHRSSLVKNLQNIYFLYEGDPVTHENVKSVDQLLSHDLIYNVSGPNYDKLKTELKNQEMATLFKDKNVDIYGVEYYHLCYLCENAERSACIYGGVTNHEGNHLEIPKKIVVKVSIDGIQSLSFDIETNKKMVTAQELDYKVRKYLTDNKQLYTNGPSKYETGYIKFIPKNKESFWFDFFPEPEFTQSKYLMIYKDNETLDSNTSQIEVYLTTK

SEQ ID NO: 6 - protein, 238 aa, Staphylococcus aureus
ESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKVDSKSVKIEVHLTTKNG

SEQ ID NO: 7 - protein, 239 aa, Staphylococcus aureus
ESQPDPTPDELHKSSEFTGTMGNMKYLYDDHYVSATKVMSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG

SEQ ID NO: 8 - protein, 239 aa, Staphylococcus aureus
ESQPDPMPDDLHKSSEFTGTMGNMKYLYDDHYVSATKVKSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLVRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG

SEQ ID NO: 9 - protein, 218 aa, Staphylococcus aureus
QGDIGVGNLRNFYTKHDYIDLKGVTDKNLPIANQLEFSTGTNDLISESNNWDEISKFKGKKLDIFGIDYNGPCKSKYMYGGATLSGQYLNSARKIPINLWVNGKHKTISTDKIATNKKLVTAQEIDVKLRRYLQEEYNIYGHNNTGKGKEYGYKSKFYSGFNNGKVLFHLNNEKSFSYDLFYTGDGLPVSFLKIYEDNKIIESEKFHLDVEISYVDSN

SEQ ID NO: 10 - protein, 208 aa, Staphylococcus aureus
TDLALANAYGQYNHPFIKENIKSDEISGEKDLIFRNQGDSGNDLRVKFATADLAQKFKNKNVDIYGASFYYKCEKISENISECLYGGTTLNSEKLAQERVIGANVWVDGIQKETELIRTNKKNVTLQELDIKIRKILSDKYKIYYKDSEISKGLIEFDMKTPRDYSFDIYDLKGENDYEIDKIYEDNKTLKSDDISHIDVNLYTKKKV

SEQ ID NO: 11 - protein, 206 aa, Staphylococcus aureus
KKDISNVKSDLLYAYTITPYDYKDCRVNFSTTHTLNIDTQKYRGKDYYISSEMSYEASQKFKRDDHVDVFGLFYILNSHTGEYIYGGITPAQNNKVNHKLLGNLFISGESQQNLNNKIILEKDIVTFQEIDFKIRKYLMDNYKIYDATSPYVSGRIEIGTKDGKHEQIDLFDSPNEGTRSDIFAKYKDNRIINMKNFSHFDIYLEK

SEQ ID NO: 12 - protein, 214 aa, Staphylococcus aureus
GTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 13 - protein, 215 aa, Staphylococcus aureus
SSTALNNMKHSYADKNPIIGENKSTGDQFLENTLLYKKFFTDLINFEDLLINFNSKEMAQHFKSKNVDVYPIRYSINCYGGEIDRTACTYGGVTPHEGNKLKERKKIPINLWINGVQKEVSLDKVQTDKKNVTVQELDAQARRYLQKDLKLYNNDTLGGKIQRGKIEFDSSDGSKVSYDLFDVKGDFPEKQLRIYSDNKTLSTEHLHIDIYLYEK

SEQ ID NO: 14 - protein, 215 aa, Staphylococcus aureus
QRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 15 - protein, 221 aa, Staphylococcus aureus
SSITLNNLRHIYFFNEKGISEKIMTEDQFLDYTLLFKSFFISHSQYNDLLVQFDSKETVNKFKGKQVDLYGSYYGFQCSGGKPNKTACMYGGVTLHENNQLYDTKKIPINLWIDSIRTVVPLDIVKTNKKKVTIQELDLQARYYLHKQYNLYNPSTFDGKIQKGLIVFHTSKEPLVSYDLFNVIGQYPDKLLKIYQDNKIIESENMHIDIYLYTSLIVLIS

SEQ ID NO: 16 - protein, 217 aa, Staphylococcus aureus
DIGIDNLRNFYTKKDFINLKDVKDNDTPIANQLQFSNESYDLISESKDFNKFSNFKGKKLDVFGISYNGQCNTKYIYGGITATNEYLDKPRNIPINIWINGNHKTISTNKVSTNKKFVTAQEIDIKLRRYLQEEYNIYGHNGTKKGEEYGHKSKFYSGFNIGKVTFHLNNNDTFSYDLFYTGDDGLPKSFLKIYEDNKTVESEKFHLDVDISYKETI

SEQ ID NO: 17 - protein, 214 aa, Staphylococcus aureus
DVGPGNLRNFYTKYEYVNLKNVKDKNSPESHRLEYSYKNDTLYAEFDNEYITSDLKGKNVDVFGISYKYGSNSRTIYGGVTKAENNKLDSPRIIPINLIINGKHQTVTTKSVSTDKKMVTAQEIDVKLRKYLQDEFNIYGHNDTGKGKEYGTSSKFYSGFDKGSVVFHMNDGSNFSYDLFYTGYGLPESFLKIYKDNKTVDSTQFHLDVEISKR

SEQ ID NO: 18 - protein, 214 aa, Staphylococcus aureus
VLNLRNYYGSYPIEDHQSINPENNHLSHQLVFSMDNSTVTAEFKNVDDVKKFKNHAVDVYGLSYSGYCLKNKYIYGGVTLAGDYLEKSRRIPINLWVNGEHQTISTDKVSTNKKLVTAQEIDTKLRRYLQEEYNIYGFNDTNKGRNYGNKSKFSSGFNAGKILFHLNDGSSFSYDLFDTGTGQAESFLKIYNDNKTVETEKFHLDVEISYKDES

SEQ ID NO: 19 - protein, 209 aa, Staphylococcus aureus
LEVDNNSLLRNIYSTIVYEYSDTVIDFKTSHNLVTKKLDVRDARDFFINSEMDEYAANDFKAGDKIAVFSVPFDWNYLSKGKVTAYTYGGITPYQKTSIPKNIPVNLWINRKQIPVPYNQISTNKTTVTAQEIDLKVRKFLIAQHQLYSSGSSYKSGKLVFHTNDNSDKYSLDLFYTGYRDKESIFKVYKDNKSFNIDKIGHLDIEIDS

SEQ ID NO: 20 - protein, 208 aa, Staphylococcus aureus
LEVDNNSLLRNIYSTIVYEYSDIVIDFKTSHNLVTKKLDVRDARDFFINSEMDEYAANDFKTGDKIAVFSVPFDWNYLSKGKVTAYTYGGITPYQKTSIPKNIPVNLWINGKQISVPYNEISTNKTTVTAQEIDLKVRKFLIAQHQLSSGSSYKSGRLVFHTNDNSDKYSFDLFYVGYRDKESIFKNYKDNKSFNIDKIGHLDIEIDS

SEQ ID NO: 21 - protein, 210 aa, Staphylococcus aureus
DENLKDLKRSLRFAYNITPCDYENVEIAFVTTNSIHINTKQKRSECILYVDSIVSLGITDQFIKGDKVDVFGLPYNFSPPYVDNIYGGIVKHSNQGNKSLQFVGILNQDGKETYLPSEVVRIKKKQFTLQEFDFKIRKFLMEKYNIYDSESRYTSGSLFLATKDSKHYEVDLFNKDDKLLSRDSFFKRYKDNKIFNSEEISHFDIYLKTY

SEQ ID NO: 22 - protein, 202 aa, Staphylococcus aureus
YNTTNRHNLESLYKHDSNLIEADSIKNSPDIVTSHMLKYSVKDKNLSVFFEKDWISQEFKDKEVDIYALSAQEVCECPGKRYEAFGGITLTNSEKKEIKVPVNVWDKSKQQPPMFITVNKPKVTAQEVDIKVRKLLIKKYDIYNNREQKYSKGTVTLDLNSGKDIVFDLYYFGNGDFNSMLKIYSNNERIDSTQFHVDVSIS

SEQ ID NO: 23 - protein, 205 aa, Staphylococcus aureus
NIKDVKLQLNYAYEIIPVDYTNCNIDYLTTHDFYIDISSYKKKNFSVDVEVESYITTKFTKNQKVNIFGLPYIFTRYDVYYIYGGVTPSVNSNENSKIVGNLLIDGVQQKTLINPIKIDKPIFTIQEFDFKIRQYLMQTYKIYDPNSPYIKGQLEIAINGNKHESFNLYDATSSSTRSDIFKKYKDNKTINMKDFSHFDIYLWTK